(19) United States
(12) Patent Application Publication
(10) Pub. No.: US 2012/0021923 A1
(43) Pub. Date: Jan. 26, 2012

(54) METHODS AND SYSTEMS TO DIAGNOSE A CONDITION IN AN INDIVIDUAL

(76) Inventors: Christopher H. CONTAG, Stanford, CA (US); Rajesh R. Shinde, Sunnyvale, CA (US); Julie Perkins, Alameda, CA (US)

(21) Appl. No.: 13/101,013

(22) Filed: May 4, 2011

Related U.S. Application Data
(63) Continuation-in-part of application No. 13/037,163, filed on Feb. 28, 2011.
Application No. 13/037,163 is a continuation-in-part of application No. 13/037,106, filed on Feb. 28, 2011.
(60) Provisional application No. 61/331,047, filed on May 4, 2010.
(60) Provisional application No. 61/331,041, filed on May 4, 2010.

(51) Int. Cl. C40B 30/00; C12Q 1/66; C12Q 1/37

(52) U.S. Cl. 506/7; 435/23; 435/8

(57) ABSTRACT

Provided herein are a method and system to detect an active protease in a sample, and related methods and systems to diagnose a condition in an individual, the condition being associated to abnormal protease activity in the individual.

### CROSS REFERENCE TO RELATED APPLICATIONS

This application is related and claims priority to U.S. Provisional Application entitled “A Method for Diagnosing Prostate Cancer by Measuring Prostate Specific Antigen from Serum” Ser. No. 61/331,047, filed on May 4, 2010 Docket No. IL11661, and to U.S. Provisional Application entitled “Early Disease Detection Though Ultrasensitive High-Throughput Serum Protease Assays” Ser. No. 61/331,041, filed on May 4, 2010 Docket No. IL11662, the disclosure of each of which is incorporated herein by reference in its entirety. The present application is also related and claims priority to U.S. patent application entitled “Methods and Systems for Synthesis Of D-Aminoluciferin Precursor And Related Compounds” Ser. No. 13/037,163 filed on Feb. 28, 2011, Docket No. IL12087, and U.S. patent application entitled “Methods and Systems for Synthesis of D-Aminoluciferin Precursor and Related Compounds” Ser. No. 13/037,106 filed on Feb. 28, 2011, Docket No. IL12088, the disclosure of each of which is incorporated herein by reference in its entirety. The present application is also related to U.S. patent application entitled “Method and Systems to Detect an Active Protease in a Sample” Ser. No. ______ filed on May 4, 2011 with docket number IL11661 herein also incorporated by reference in its entirety.

### STATEMENT OF GOVERNMENT GRANT

The United States Government has rights in this invention pursuant to Contract No. DE-AC52-07NA27344 between the United States Department of Energy and Lawrence Livermore National Security, LLC for the operation of Lawrence Livermore National Laboratory.

### FIELD

The present disclosure detection of one or more proteases, in particular protease biomarkers, in a sample such as a biological sample.

### BACKGROUND

High sensitivity detection of proteases and in particular protease biomarkers has been a challenge in the field of biological molecule analysis, in particular when aimed at detection of a plurality of proteases having different affinity and activities for the respective target peptides. Whether for pathological examination or for fundamental biology studies, several methods are commonly used for the detection of various classes of proteases in particular when the proteases are biomarkers.

Some of the techniques most commonly used in the laboratory for protease detection (e.g. ELISA) have provided the ability to detect proteases in biological samples—such as tissues—and are also suitable for diagnostic purposes. Particularly informative in some occurrences is a detection that takes into account both single protease data and multiple proteases data in connection with diagnosis of a condition (e.g. cancers).

However, in several occurrences, accurate detection of one or more active proteases can still be challenging, in particular, when the one or more proteases are present in low concentration and/or difficult to accurately detect due to their relevant properties (e.g. similarities with other molecule in the sample).

### SUMMARY

Provided herein, are method and systems for detecting protease activity in a sample, which allow in several embodiments sensitive detection of active proteases present at low concentration. In particular, methods and systems herein described allow detection of proteases that are associated with conditions in an individual.

According to a first aspect, a method and system to detect an active protease in a sample are described. In the method the active protease is able to specifically cleave a corresponding target peptide. The method comprises providing a substrate comprising the target peptide conjugated with a label, the substrate configured to allow release of the label upon cleavage of the target peptide by the protease, the label configured to produce a bioluminescent signal upon release from the substrate. The method further comprises contacting the sample with the substrate for a time and under condition to allow cleavage of the target peptide by the protease; detecting the bioluminescent signal and detecting the active protease in the sample based on the detected bioluminescent signal. The system comprises one or more substrates each comprising the target peptide conjugated with the label and reagents for detecting bioluminescence signal from the label.

According to a second aspect, a method and a system to diagnose a condition in an individual is described. In the method, the condition is associated to a predetermined concentration of one or more protease in an active form and the one or more active protease is able to specifically cleave a corresponding target peptide. The method comprises contacting a sample from the individual with a substrate comprising the target peptide conjugated with a label, the substrate configured to allow release of the label upon cleavage of the target peptide by the protease, the label configured to produce a bioluminescent signal upon release from the substrate. In the method the contacting is performed for a time and under condition to allow cleavage of the target peptide by the protease. The method further comprises detecting the bioluminescent signal, detecting a concentration of the active protease in the sample based on the detected bioluminescent signal, and comparing the detected concentration with the predetermined concentration to diagnose the condition in the individual. The system comprises one or more substrates each comprising the target peptide conjugated with the label and reagents for detecting bioluminescence signal from the label and a look up table comprising predetermined concentrations associated to diagnose of one or more conditions in an individual.

According to a third aspect, a method to diagnose a prostate condition in an individual is described. The method comprises detecting PSA in active form in a sample from the individual, and detecting total PSA in the sample from the individual. The method further comprises determining a ratio of active PSA/total PSA, wherein a ratio of about ≦0.1% or of about ≦1/1000 is indicative of presence of a prostate condition in the individual.

According to a fourth aspect, a support suitable to identify an active protease in a sample. The support comprises a capture agent able to specifically bind the protease in a capture agent-protease binding complex, wherein the protease in the capture agent protease binding complex is proteolytically active.

The methods and systems herein described allow in several embodiments detection of a protease with an accuracy that allows discriminating between active form and non active form of a same protein for proteases present at low concentration such as amounts in the order of picograms or lower and therefore identification of a ratio for diagnosing purposes in any case where the difference is indicative of a condition in an individual.

The methods and systems herein described allow in several embodiments detection of a protease with an accuracy that allows discriminating between protein present at a low concentration in a sample further comprising other proteins at a higher concentrations and therefore identification of the proteases in proteomics for various purposes diagnosing because before it was not possible to really discriminate]

The methods and systems herein described can be used in connection with applications wherein detection of an active protease is desired, including but not limited to medical application, biological analysis and diagnostics including but not limited to clinical applications.

The details of one or more embodiments of the disclosure are set forth in the accompanying drawings and the description below. Other features, objects, and advantages will be apparent from the description and drawings, and from the claims

### BRIEF DESCRIPTION OF THE DRAWINGS

The accompanying drawings, which are incorporated into and constitute a part of this specification, illustrate one or more embodiments of the present disclosure and, together with the detailed description and examples sections, serve to explain the principles and implementations of the disclosure.

FIG. 1 shows liquid chromatography/mass spectrometry spectra for a compound according to an embodiment herein described

FIG. 2 shows reaction schemes illustrating use of combinatorial chemistry for synthesis and screening to identify optimal substrates for PSA. FIG. 2A shows a schematic representation of a multigram scale synthesis of luciferin analogs performed to provide a substrate suitable for a method to detect one or more protease according to an embodiment herein described. FIG. 2B shows a schematic representation of a split mix approach using a solid phase combinatorial library according to an embodiment herein described. FIG. 2C shows a schematic representation of a screening of beads against PSA according to an embodiment herein described.

FIG. 3 shows a schematic representation of a bioluminescent assay based on release of D-aminoluciferin from labeled compounds according to an embodiment herein described.

FIG. 4 shows diagrams illustrating sensitivity of luciferyl substrate SKLQ-aluc. In particular FIG. 4A and FIG. 4B shows charts illustrating PSA cleavage of aluc from SKLQ-aluc after 14 hours of incubation. In FIG. 4A the y axis shows the Relative Light Units (RLU), the x axis shows the PSA concentration. In FIG. 4B the Y-axis shows the net RLU, i.e. the RLU for a sample-baseline SKLQ-aluc reading), and the x axis shows the PSA concentration.

FIG. 5 shows diagrams illustrating the kinetics of a protease activity with a fluorescent substrate according to an embodiment herein described. FIG. 5A shows a chart illustrating rate of release of fluorophore from a fluorescent substrate after cleavage by PSA at various concentrations. The enzymatic reaction was carried out using a buffer, PSA-A with 0.15 M NaCl. FIG. 5B shows a chart illustrating the velocity of release of fluorophore at different substrate concentrations of PSA. The Michelis-Menten kinetics parameters obtained for PSA for the fluorescent substrate, KGISSQY-AFC are Km=1.73 mM and Vmax=3.7 RLU/sec.

FIG. 6 shows diagrams illustrating the kinetics of a protease activity with a fluorescent substrate according to an embodiment herein described. FIG. 6A shows a chart illustrating rate of release of fluorophore from a fluorescent substrate after cleavage by PSA at various concentrations. The enzymatic reaction was carried out using a buffer, PSA-B with 1.5 M NaCl. Increasing the NaCl concentration lead to changes in the observed Michelis-Menten parameters. FIG. 6B shows a chart illustrating the velocity of release of fluorophore at different substrate concentrations of PSA. The Michelis-Menten kinetics parameters obtained for PSA from these experiments for the fluorescent substrate, KGISSQY-AFC are Km=0.47 mM and Vmax=2.33 RLU/sec. The results indicate that enzymatic efficiency of PSA is affected by the buffer in which the enzymatic reaction takes place.

FIG. 7 shows a schematic representation of approached used to detect the PSA on beads. FIG. 7A illustrates a bead based immunocapture. FIG. 7B illustrates a microplate-based immunocapture.

FIG. 8 shows a schematic representation of epitopes of PSA and the active site.

FIG. 8A shows a schematic representation of the three dimensional structure of PSA from Huhtinen et al., J. Immuno. Methods, 294, 111-122 (2004) indicating epitopes in the active form (left panel) and the inactive form (right panel) wherein the active site is indicated with a triangle. FIG. 8B shows a schematic representation of PSA from Tumor Biology—Workshop, 20, 1-12 (1999) wherein a detailed map of the epitopes further indicating the related amino acid residues is indicated in a bidimensional (left panel) and three dimensional (right panel) illustration.

FIG. 9 shows a diagram illustrating an influence of antibody binding on protease activity according to an embodiment herein described. In particular, the Y axis shows the AFC released by a suitable substrate and the X axis shows different sample with concentration of PSA, PSA/Mab30 and PSA/MAB26 as indicated.

FIG. 10 shows diagrams illustrating immunocapture and activity studies of a protease according to embodiments herein described. FIG. 10A shows a diagram illustrating the percentage recovery of PSA by Mab-26 in presence or absence of BSA as indicated. The Y axis shows the RLU the X axis shows concentration of Mab26 incubated with beads. FIG. 10B shows a diagram illustrating the percentage recovery of PSA by Mab-30 in presence or absence of BSA as indicated. The Y axis shows a RLU the X axis shows concentration of Mab30 incubated with beads.

FIG. 11 shows diagrams illustrating immunocapture and activity studies of a protease according to embodiments herein described. FIG. 11A shows a diagram illustrating the PSA recovery by Mab-26 and Mab-30 compared with unbound PSA and a control at different Mab concentrations as indicated in the chart. The Y axis shows the RLU the X axis shows concentration of PSA incubated with beads FIG. 11B shows a diagram illustrating PSA recovery of by Mab 26 and Mab30 with unbound PSA and a control. The Y axis shows the RLU the X axis shows concentration of PSA incubated with beads.

FIG. 12 shows diagrams illustrating active PSA detection using different target peptides according to an embodiment here described. FIG. 12A shows a diagram illustrating active PSA detected in buffer using SKLQ-aluc peptide. The y axis shows the RLU/sec as measured from a luminometer and the x axis shows corresponding active PSA detected. FIG. 12B shows a diagram illustrating active PSA detected in buffer using KGISSQY-aluc peptide. The y axis shows the RLU/sec from a luminometer and the x axis shows corresponding active PSA detected.

FIG. 13 shows diagrams illustrating active PSA detection using different target peptides according to an embodiment here described. FIG. 13A shows a diagram illustrating active PSA detected in serum using KGISSQY-aluc peptide. The y axis shows the RLU/sec and the x axis shows corresponding active PSA detected. FIG. 13B shows a diagram illustrating active PSA detected in serum using SKLQ-aluc peptide. The y axis shows the RLU/sec and the x axis shows corresponding active PSA detected. The experiments were accomplished using beads coated with anti-PSA Mab.

FIG. 14 shows diagrams from R. Etzioni et al., Nature Reviews Cancer, 3, 1-10, (2003), illustrating the impact on the stage of detection on the efficacy of treatment in various forms of cancer.

FIG. 15 shows a schematic representation of the categorization of plasma protein from J. Jacobs et al., J. Proteome Research, 4, 1073-85 (2005).

FIG. 16 shows a schematic representation illustrating PSA as a biomarker.

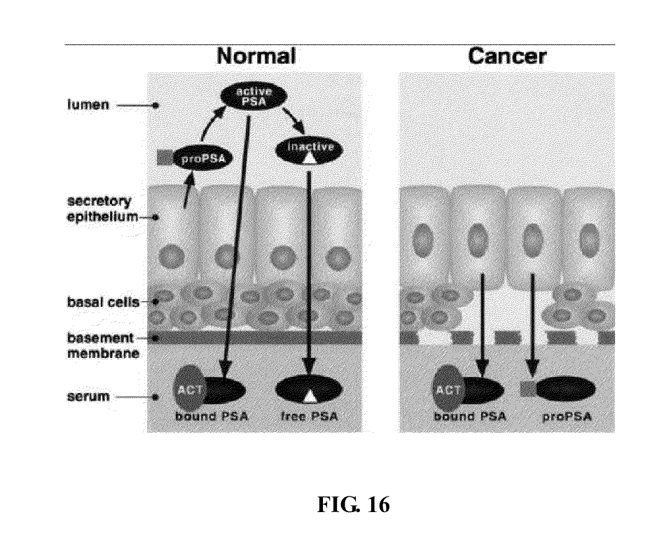

FIG. 17 shows a schematic representation of the various PSA isoforms and the respective relevance as biomarker. In particular, FIG. 17A shows prostate-specific antigen (PSA) subforms and interactions. FIG. 17B shows forms of free PSA in serum. FIG. 17C shows association of free PSA forms with prostate cancer.

FIG. 18 shows a schematic representation of an assay performed to detect a protease biomarker for diagnosing purposes according to an embodiment herein described.

FIG. 19 shows a schematic representation of a bioluminescent assay based on release of D-aminoluciferin from labeled compounds according to an embodiment herein described.

### DETAILED DESCRIPTION

Provided herein, are methods and systems to detect a protease activity and active proteases in a sample and a related method of diagnosing a condition in an individual.

The terms “detect” or “detection” as used herein indicates the determination of the existence, presence or fact of a target in a limited portion of space, including but not limited to a sample, a reaction mixture, a molecular complex and a substrate. The “detect” or “detection” as used herein can comprise determination of chemical and/or biological properties of the target, including but not limited to ability to interact, and in particular bind, other compounds, ability to activate another compound and additional properties identifiable by a skilled person upon reading of the present disclosure. The detection can be quantitative or qualitative. A detection is “quantitative” when it refers, relates to, or involves the measurement of quantity or amount of the target or signal (also referred as quantitation), which includes but is not limited to any analysis designed to determine the amounts or proportions of the target or signal. A detection is “qualitative” when it refers, relates to, or involves identification of a quality or kind of the target or signal in terms of relative abundance to another target or signal, which is not quantified.

The term “protease” as used herein indicates any enzyme that conducts proteolysis, that is, begins protein catabolism by hydrolysis of the peptide bonds that link amino acids together in the polypeptide chain forming the protein. Proteases are typically biomolecules, which in some cases, can be used as biomarkers. The term “biomolecule” as used herein indicates a substance, compound or component associated to a biological environment. The term “biomarker” indicates a biomolecule that is associated to a specific state of a biological environment including but not limited to a phase of cellular cycle, health and disease state. The presence, absence, reduction, upregulation of the biomarker is associated to and is indicative of a particular state. The “biological environment” refers to any biological setting, including, for example, ecosystems, orders, families, genera, species, subspecies, organisms, tissues, cells, viruses, organelles, cellular substructures, prions, and samples of biological origin.

Proteases comprise enzymes occurring naturally in all organisms. In some embodiments herein described, proteases comprise enzymes that can be found in various bodily fluids and/or in other samples. For example proteases can be found in blood and various glands and organs including salivary glands, thyroid, thymus, prostate, brain, skin, trachea, lung, kidney, pancreas, uterus, colon, breast, ovary, and testis. These enzymes are typically involved in a multitude of physiological reactions from simple digestion of food proteins to highly-regulated cascades (e.g., the blood-clotting cascade, the complement system, apoptosis pathways, and the invertebrate prophenoloxidase-activating cascade) and can be used as a biomarker of one or more biological states including biological states associated to one or more condition in an individual. Proteases in the sense of the present disclosure indicate enzyme that can break specific peptide bonds (limited proteolysis), depending on the amino acid sequence of a protein. The protease activity can result in a destructive change, abolishing a protein's function or digesting it to its principal components; it can be an activation of a function, or it can be a signal in a signaling pathway .

The term “protein” as used herein indicates a polypeptide with a particular secondary and tertiary structure that can interact with another analyte and in particular, with other biomolecules including other proteins, DNA, RNA, lipids, metabolites, hormones, chemokines, and small molecules. The term “polypeptide” as used herein indicates an amino acid polymer of any length including full length proteins and peptides, as well as analogs and fragments thereof. A polypeptide of three or more amino acids is also called a protein oligomer, peptide or oligopeptide. In particular, the terms “peptide” and “oligopeptide” usually indicate a polypeptide with less than 50 amino acid monomers. As used herein the term “amino acid”, “amino acidic monomer”, or “amino acid residue” refers to any of the twenty naturally occurring amino acids, non-natural amino acids, and artificial amino acids and includes both D an L optical isomers. In particular, non-natural amino acids include D-stereoisomers of naturally occurring amino acids (these including useful ligand building blocks because they are not susceptible to enzymatic degradation). The term “artificial amino acids” indicate molecules that can be readily coupled together using standard amino acid coupling chemistry, but with molecular structures that do not resemble the naturally occurring amino acids. The term “amino acid analog” refers to an amino acid in which one or more individual atoms have been replaced, either with a different atom, isotope, or with a different functional group but is otherwise identical to original amino acid from which the analog is derived. All of these amino acids can be synthetically incorporated into a peptide or polypeptide using standard amino acid coupling chemistries (see e.g. Lam, K. S. et al., Chem. Rev., VOL 97, page 411-448 (2007) incorporated herein by reference in its entirety).

Methods and systems herein described are directed to detection of an active protease in a sample. The term “active protease” as used herein indicates an isoform of a protease that is capable to carry out enzymatic cleavage of a substrate for the protease. Enzymatic cleavage can be detected through various techniques and procedures identifiable by a skilled person comprising for example, detection of the cleavage product, detection of isoforms associated to the ability to carry out the enzymatic cleavage such as the procedures described in Niemela et al Clinical Chemistry 48:8 1257-1264 (2002) and other references identifiable by a skilled person that typically make use of labels.

Proteases in the sense of the present description comprise serine proteases, threonine proteases, cysteine proteases, aspartate proteases and glutamic acid proteases and are typically capable to specifically cleave one or more target peptides.

In particular, in some embodiment, the protease detectable with methods and systems here described is prostate-specific antigen (PSA) and the target peptide can comprise one or more of KGISSQY (SEQ ID NO: 1), SRKSQQY (SEQ ID NO: 2), GQKGQHY (SEQ ID NO: 3), EHSSKLQ (SEQ ID NO: 4), QNKISYQ (SEQ ID NO: 5), ENKISYQ (SEQ ID NO: 6), ATKSKQH (SEQ ID NO: 7), KGLSSQC (SEQ ID NO: 8), LGGSQQL (SEQ ID NO: 9), QNKGHYQ (SEQ ID NO: 10), TEERQLH (SEQ ID NO: 11), GSFSIQH (SEQ ID NO: 12), HSSKLQ (SEQ ID NO: 13), SKLQ (SEQ ID NO: 14), KLQ (SEQ ID NO: 15), LQ (SEQ ID NO: 16), and additional peptides described in S. R. Denmeade et al. (Cancer research, 1997, vol. 57, page 4924-4930) incorporated herein by reference in its entirety and/or additional target peptides identifiable by a skilled person.

The term “target peptide” as used herein indicates an amino acid sequence that is specifically recognized and subsequently cleaved by a protease. The wording “specific” “specifically” or “specificity” as used herein with reference to the binding of a first molecule to second molecule refers to the recognition, contact and formation of a stable complex between the first molecule and the second molecule, together with substantially less to no recognition, contact and formation of a stable complex between each of the first molecule and the second molecule with other molecules that may be present. Exemplary specific bindings are antibody-antigen interaction, cellular receptor-ligand interactions, polynucleotide hybridization, enzyme substrate interactions etc. The term “specific” as used herein with reference to a molecular component of a complex, refers to the unique association of that component to the specific complex which the component is part of. By “stable complex” is meant a complex that is detectable and does not require any arbitrary level of stability, although greater stability is generally preferred. The term “specific” “specifically” “specificity” or “selective” as used herein with reference to a chemical or biological activity of a first molecule upon a second molecule refers to the ability of the first molecule to direct the activity towards the second molecule, together with substantially less to no activity between the first molecule upon a third molecules that may be present. Given a certain protease of interest, which can comprise a plurality of proteases of interest, a target peptide has typically certain properties including, for example, a certain specificity and a certain efficiency with respect to the protease of interest. Those properties are detectable with progress curve analysis (see e.g. Example 3 below) and additional methods identifiable by a skilled person.

In methods and systems herein described, some embodiments, a target peptide is conjugated with a label to form a suitable substrate which is provided for use in methods and systems herein described.

The term “conjugate” or “couple” as used herein indicates formation of a covalent bond between two compounds which encompasses either direct or indirect conjugation such that for example where a first compound is directly bound to a second compound or material, and the embodiments wherein one or more intermediate compounds, and in particular molecules, are disposed between the first compound and the second compound or material.

The terms “label” and “labeled molecule” as used herein as a component of a complex or molecule referring to a molecule capable of detection, including but not limited to radioactive isotopes, fluorophores, chemiluminescent dyes, chromophores, enzymes, enzymes substrates, enzyme cofactors, enzyme inhibitors, dyes, metal ions, nanoparticles, metal sols, ligands (such as biotin, avidin, streptavidin or haptens) and the like. The term “fluorophore” refers to a substance or a portion thereof which is capable of exhibiting fluorescence in a detectable image. As a consequence, the wording “labeling signal” as used herein indicates the signal emitted from the label that allows detection of the label, including but not limited to radioactivity, fluorescence, chemiluminescence, production of a compound in outcome of an enzymatic reaction and the like.

In methods and systems herein described, the substrate is in particular configured to allow release of the label upon cleavage of the target peptide by the protease, the label configured to produce a signal upon release from the substrate. Possible configurations are identifiable by a skilled person on view of the target peptide, related cleavage site for one or more proteases to be detected and specific label selected according to the experimental design. Suitable substrates can be provided in view of the protease to be detected, related properties of the protease (e.g. structure, concentrations, similarities with other proteins present in the sample etc.) the results to be detected (e.g. protease activity profile of the protease alone or in combination with other proteases) and additional parameters identifiable by a skilled person.

In particular, in some embodiments, following identification of the one or more protease to be detected in active form, a suitable substrate can be provided by selecting a suitable target peptide, selecting a suitable label, and conjugating or coupling the target peptide with the label.

In some embodiments, selecting a suitable target peptide can be performed based on the properties of the target peptide, and in particular the efficiency and specificity of the target peptide with respect to the protease to be detected and/or the ability of the peptide to be conjugated with a suitable label. For example, in embodiments where the method is directed to distinguishing one type of protease of interest from other types of proteases that are present together in a sample, selecting a target peptide can be performed to select the target peptide that has a high specificity to the protease of interest. In embodiments wherein the protease comprises one or more active proteases that are homologous or otherwise belong to a proteases family, selecting a target peptide that can serve as a general substrate for the one or more protease or the protease family can be desired based on the specificity for the protease family. In some embodiments, suitable target peptides can be selected using libraries expressing various peptides that are then selected based on the desired specificity, efficiency and/or additional properties of the target peptide that are functional to the desired protease to be detected, the label to be used and/or the specific experimental design. In particular, in some embodiments, the target peptide can be selected by combinatorial chemistries, phage display libraries or other method identifiable to a skilled person in the art. Providing a target peptide can be performed for example by retrieving the target peptide by commercial sources, performing organic synthesis of the target peptide or by additional method identifiable to a skilled person in the art.

In some embodiments, selecting a suitable label is performed based on the structural properties of the label and/or detectability of the labeling signal in view of the protease concentration to be detected, suitable target and corresponding configuration of the substrate as will be understood by a skilled person. Additional factors comprise the stability of a substrate comprising the target peptide in an assay environment (e.g. serum) and the properties of side chains of the peptide and additional factors identifiable by a skilled person upon reading of the present disclosure. In some embodiments, selecting a suitable label can be performed based on the desired type of labeling signal to be detected in the context of the method, the steps of conjugating the label with a desirable target peptide, the stability of a substrate comprising the label in an essay environment and reactivity of the label with other entities present in the sample.

In some embodiments, conjugating a target peptide and in particular a preselected target peptide, with a suitable label, in particular a preselected label can be performed with chemical methods directed to provide a controlled linkage between the target peptide and the label in a configuration that will allow release of the label upon cleavage of the peptide by the protease to be tested.

In some embodiments, the target peptide can comprise any peptide that is able to be specifically cleaved by the one or more proteases at issue and is also capable of being joined to a suitable bioluminescent label herein described.

In some embodiments, the target peptide can comprise one or more amino acids with each amino acid having a side chain, wherein the peptide have been found to be easily conjugated with a desirable label to provide a suitable substrate despite the expected modifications in biodistribution (pharmacokinetics/pharmacodynamics). In particular, in some embodiments, the amino acid is an amino acid having a hydrophobic side chain (including for example Alanine, Isoleucine, Leucine, Methionine, Phenylalanine, Tryptophan, Tyrosine and Valine). In some embodiments, the amino acid is an amino acid having a non-polar uncharged side chain (including for example Serine, Threonine, Asparagine and Glutamine). In some embodiments, the amino acid is an amino acid having an electrically charged side chain (including for example Arginine, histidine and Lysine, Aspartic acid and Glutamic acid).

In some embodiments, conjugating a target peptide with a label can be performed by combinatorial chemistry to provide a suitable substrate for a protease of interest. In particular, in some embodiments, conjugating can be performed through a split mix approach using a solid phase combinatorial library (see Example 2).

In some embodiments, conjugating a target peptide with a label can be performed through a series of organic synthesis steps such as synthesizing an active precursor molecule and subsequent synthesizing the substrate using the precursor molecule as a starting reactant (see Example 1).

In some embodiments, conjugating a target peptide with a label can be performed using biotinylation reagents to chemically tag the target peptide at particular functional groups of the side chains of the target peptide.

In other embodiments, conjugating a target peptide with a label can be performed using enzyme labeling reagents, such as Horseradish peroxidase (HRP), alkaline phosphatase (AP).

In some embodiments, conjugating a target peptide to a label can be performed using fluorescent labeling reagents such as fluorescent dyes for labeling amines, sulfhydryls and other functional groups of the side chains of the target peptide.

In some embodiments, conjugating a target peptide to a label can be performed using iodine labeling reagents, such as tyrosine addition compounds and tyrosyl activation vessels that efficiently label the target peptide with radioactive iodine (e.g. I125), during a process known as iodination.

In some embodiments, conjugating a target peptide to a label can be performed using metabolic labeling reagents such as bioorthogonal azide-modified amino acids, sugars and other compounds for metabolic incorporation into proteins and macromolecules to enable labeling and chemoselective conjugate with alkyne or phosphine reagents via Staudinger ligation chemistry.

In some embodiments of the methods and systems herein described, target peptides are conjugated with a bioluminescent label which provides a bioluminescent signal. The term “bioluminescence” as used herein indicates the production and emission of light by one or compounds originated from living organisms. The term “bioluminescent label” as used herein indicates a compound when undergoes chemical changes or reactions, produces and emits light and comprises compounds typically originated from living organisms or analogues and variants thereof. For example, a bioluminescent label can be a substrate molecule of a light-emitting enzyme, such as luciferin, which represents a class of light-emitting biological pigments found in luminous organism, including fireflies, bacteria, squid and jellyfish, oxidized by a luciferase or other photoproteins (e.g. aequorin) to form oxyluciferin accompanied by release of a photon.

In some embodiments, the bioluminescent label is D-luciferin or a functional derivative or analog thereof. In particular, in some embodiments, the bioluminescent label can be an aminoluciferyl label, wherein the term “aminoluciferyl label” as used herein indicates a derivative of D-luciferin, the natural substrate of luciferase, including but not limited to, aminoluciferin. The term “aminoluciferin (aLuc)” refers to a luciferin with its 6-position hydroxyl group substituted with an amino group. This modification allows aLuc to form peptide (amide) bonds with a peptide, while at the same time retaining the transport and bioluminescent properties of luciferin, resulting in a molecule called peptide-aminoluciferin. Several methods and systems for conjugating of a aminoluciferin into a peptide is described in U.S. patent application Ser. No. 13/037,163 and U.S. patent application Ser. No. 13/037,106 incorporated herein by reference in its entirety (See e.g. Example 1).

In some embodiments, the aminoluciferyl label can comprise a suitable amino acid conjugated with the D-luciferin. In particular, in some embodiments the amino acid is one of the naturally occurring amino acids, including Isoleucine, Alanine, Leucine, Asparagine, Lysine, Aspartic Acid, Methionine, Cysteine, Phenylalanine, Glutamic Acid, Threonine, Glutamine, Tryptophan Glycine, Valine, Proline, Serine, Tyrosine, Arginine, Histidine, and additional amino acids such as Selenocysteine Ornithine, and Taurine. In some embodiments, the amino acid comprises a side chain hydrophobic, hydrophilic, acid or basic in nature.

In some embodiments, the aminoluciferyl peptide comprises luciferyl-tyrosine (see Examples 1 and 2).

In an embodiment, providing a substrate for a protease to be detected can be performed through screening a solid phase combinatorial library of many candidate substrates in a protease activity assay using recombinantly produced and purified protease of interest (See Example 2).

In some embodiments, the label signal can be selected to have a labeling signal with a low intrinsic background in cells and tissues, sometimes in contrast to fluorescent-based detection, and in particular antofluorescence.

In some embodiments, the method comprises contacting the sample with the substrate for a time and under condition to allow cleavage of the target peptide by the protease; detecting the bioluminescent signal and detecting the active protease in the sample based on the detected bioluminescent signal.

The term “sample” as used herein indicates a limited quantity of something that is indicative of a larger quantity of that something, including but not limited to fluids from a biological environment, specimen, cultures, tissues, commercial recombinant proteins, synthetic compounds or portions thereof. In particular, in some embodiments, the sample is serum from an individual.

In some embodiments, the contacting can be performed in free solution. Particularly, according to some embodiments, the contacting can be performed by adding directly to the sample a protease specific target peptide conjugated to an aminoluciferyl or other appropriate label with the adding performed in a solution, and performing the contacting for a sufficient incubation time to allow cleavage of the label from the substrate.

In some embodiments, the solution comprises one or more components that can facilitate the enzymatic activity of the protease upon the substrate under appropriate conditions identifiable by a skilled person. For example, in some embodiments, the solution can comprise a suitable combination of HEPES, PBS, Tris-Acetate, Gly-gly and NaCl. IN particular, NaCl can be comprised in suitable concentration such as 0.15 M-1.5 M NaCl. In some embodiments, the solution can further comprise BSA (see e.g. Example 4).

In some embodiments, the contacting can be performed on a solid support. The term “support” as used herein indicates an inert substance that is in contact with the sample and the substrate during the contacting, including but not limited to, magnetic material, polymers, such as sepharose, inner surface of a tube, vessel, microtiterplate or other platforms (see e.g. Example 5) where the contacting takes place.

The term “platform” as used herein indicates a support comprising one or more reaction units where one or more chemical reactions under consideration for the methods and systems herein described, such as the cleavage of the target peptide by the protease take place, including but not limited to, reaction tubes, vessels, wells and plates.

The term “inert substance” as used herein indicates a material that does not participate in chemical reaction with any other components under consideration for the methods and systems herein described, including but not limited to polymers, such as sepharose, agar, nitrocellulose and magnetic beads.

In some embodiments, the method can further comprise separating the protease from the sample before the contacting with the substrate.

In some embodiments, the separating can be performed by coating the inner surface of a platform with a layer comprising a suitable capture agent, contacting the sample with the coated inner surface for a time and under condition to allow association of the protease to the capture agent, and recovering the protease associated capture agent from the sample.

In other embodiments, the platform further comprises an inert substance, and the separating comprises coating the inert substance with a layer comprising a suitable capture agent, contacting the sample with the coated inert substance for a time and under condition to allow association of the protease to the capture agent, and recovering the protease associated capture agent from the sample.

The term “coating” as used herein indicates the formation of a layer attached to the surface of the solid support. The attachment of the layer to the surface can be direct or indirect physical connection and/or through direct or indirect interaction/bonding of molecules. The attachment of the layer to the surface can be either permanent or reversible. The layer can contain one or more components, including but not limited to capture agents such as antibody, protein A, polypeptides and polynucleotides.

The term “capture agent” as used herein indicates a compound that can specifically bind to a target and particularly to a protease. Suitable capture agents can include but are not limited to organic molecules, such as polypeptides, polynucleotides and other non polymeric molecules that are identifiable to a skilled person. In particular, in some embodiments capture agents can comprise aptamers capable of specifically binding the protease at issue.

In some embodiments, the separating of the protease can be performed using other protein separation methods that are identifiable by a skilled person, and include but are not limited to size-exclusion chromatography (SEC) and isoelectric focusing (IEF).

The term “separate” as used herein indicates setting, keeping apart or making a distinction between an item and another, and in particular between a target and another analyte which is not of interest, and includes sorting a plurality of proteases of interest. The term “sort” as used herein indicates to set a group set up on the basis of any characteristic in common. In particular, the capture agent can be used to separate one or more proteases and/or sorting a plurality of proteases in a sample.

In embodiments where the separating of the protease is performed, a separated protease population present in the sample can be captured for further assays.

In some embodiments, capture agents suitable to separate the protease can be preselected to minimize interference between the capture agents and the active site of the protease (see e.g. Example 6). In particular pre-selection can be performed to maximize the capturing of active form of a protease in view of the protease structure (see e.g. Example 6). Additional features of the capture agents such as reaction conditions, effective concentrations, and additional features identifiable by a skilled person upon reading of the present disclosure can be tested to select the proper capture agent (see Examples 7-10 and 12).

In methods and systems herein described, following the contacting the bioluminescent signal is detected. In particular, suitable reagents for detecting the signal are contacted with the mixture. For example in case of aminoluciferyl label a luciferase cocktail can be added and bioluminescence can be detected using appropriate techniques.

As used herein, the term “luciferase” refers to one or more oxygenases that catalyze a light emitting reaction. Thus, luciferase refers to an enzyme or photoprotein that catalyzes a reaction that produces bioluminescence. In general in embodiments, wherein the label is luciferin, aminoluciferin or another analog thereof, luciferase can be used for detection. For example, when the substrate is, an aminoluciferyl peptide, reagents for detecting bioluminescence signal from the label can comprise luciferase.

Suitable luciferases in methods and systems herein described comprise recombinant or naturally occurring luciferases, as well as a variant or mutant thereof. Exemplary naturally occurring luciferases comprise luciferases found among marine arthropods, firefly luciferase, click beetle luciferase, and railroad worm luciferase. In particular, exemplary luciferases comprise a luciferase photoprotein and more particularly the aequorin photoprotein. Naturally occurring luciferases further comprise for example the luciferase produced by beetle, such as the North American firefly, Photinus pyralis. Exemplary variants comprise a variant produced by mutagenesis that has one or more properties, such as thermal stability, that differ from the naturally-occurring protein while substantially retaining the ability to catalyze the light emitting reaction. Luciferase catalyzes the conversion of luciferin, in the presence of oxygen, Mg2+, and ATP, to oxyluciferin accompanied by release of a photo. The light-emitting reaction of luciferase-luciferin has been adapted for bioluminescence imaging technology for preclinical molecular imaging.

In some embodiments, the label signal can be detected in free solution using a luminometer, such as a UV detector, or other methods or devices identifiable to a skilled person. In other embodiments, the label signal can be performed on a solid support, wherein the light emitting label is captured onto a solid support, such as a flat surface, and the signal is detected using a light-sensitive film, a scanner or other methods or devices identifiable to a skilled person.

In exemplary embodiments, a luciferase from fireflies or click beetle (CBR) can be used for detection and the label comprises luciferin or a synthetic analog thereof. An exemplary synthetic analog of luciferin is aminoluciferin (aluc), wherein the —OH group on luciferin is replaced by an amine group, which results in a label comprising an unnatural amino acid with an amine group on one end and a carboxylic acid group on the other end. In some of those embodiments the aLuc can be conjugated to an amino acid in a peptide sequence. The proteases that cleave any amide bonds at the N-terminus of a particular peptide sequence can then release free aluc which can then react with luciferase and release light. In exemplary embodiments wherein the protease is PSA, 2 peptide sequences, can possibly be used (e.g. SKLQ-aluc and KGISSQY-aluc see Examples section). In those embodiments, PSA will have a preference to cleave at the N-terminus of Q and Y to release aluc.

In some embodiments, the method further comprises washing the recovered capture agent to remove other entities present in the sample before the detecting of the bioluminescent signal.

In some embodiments, the method further comprises filtering the sample contacted with the substrate to collect the released label in a filtrate before detecting the bioluminescent signal. For example, in embodiments wherein the substrates aminoluciferyl peptides exhibit a significant background, as can be the case for certain aminoluciferyl sequences, then the incubation mixture can be filtered such that the bigger peptide or uncleaved peptide is retained whereas aminoluciferin is collected as the filtrate and mixed with the luciferase-cocktail and quantified.

In some embodiments, a luciferin regenerating enzyme (LRE) can be used to recycle oxyluciferin the product of the aminoluciferin thus providing a long lasting signal, which is longer than about 15 minutes and in particular can be from about 3 to about 5 hours duration. The LRE can be added to the luciferase cocktail for optimum and longer lasting bioluminescence signal.

In some embodiments, methods and systems herein described allow detection that can be very sensitive and in particular detect the functionality of low amounts of proteases such as PSA. In some embodiments, methods and systems herein described can detect a protease present in the sample at a concentration of about ≦1 nmol/ml, and more particularly at a concentration of about ≦0.1 pmole/ml, even more particularly of about ≦0.1 femtomole/ml and even more particularly at a concentration of about 1 to about 10 atta moles. In certain embodiments, wherein detection of a concentration as low as 1 to 10 attograms per ml can be achieved, the label can be a bioluminescent label.

In some embodiments, the proteases whose active form is detected can have more than one isoforms. In some of those embodiments only some of the isoforms are active and capable of proteolytic cleavage of substrates. In those embodiments production of active isoform can be performed starting from an inactive isoform through steps of removing or detaching of a pro-sequence or inhibitor sequence, or additional modifications identifiable by a skilled person. In some cases, the active and inactive isoforms of a protease can be very similar in primary and/or secondary and tertiary structure. Therefore, distinguishing and/or separation among the various isoforms based on their structural differences (e.g. immunoprecipitation or chromatography) can be challenging.

In some embodiments, activity of a protease can be highly regulated in an organism, possibly through regulating production and turnover of certain types of proteases (e.g. from inactive to active isoforms). For example a possible mechanism for regulating protease activity is through regulating the amount of protease present in an active or inactive form, including regulating coupling of the protease to the inhibitors. In particular, in some instances anti protease proteins (such as anti-trypsins and anti-chymotrypsins) can complex with the protease, (e.g. trypsins and chymotrypsins) and deactivate them. PSA is a type of chymotrypsin and is complexed with the anti-chymotrypsin and sometimes anti-trypsins.

In some embodiments, active proteases detectable with methods and systems herein described can be comprised in an environment, especially of biological nature in a very low amount within a picogram range or even lower. In exemplary embodiments, wherein the protease is PSA, detection can be performed a PSA concentration of about ≦0.1 pmoles/ml.

In some embodiments, the protease is a biomarker and detecting an amount of active protease and a determining a corresponding protease activity can be used as an indicator of a biological state and in particular, biological conditions, including for example diseases.

In an embodiment, the protease is prostate-specific antigen (PSA). The term “PSA” as used herein indicates a serine protease secreted by both normal prostate glandular cells and prostate cancer cells. PSA exists in serum predominantly as a complex with the protease inhibitor alpha-1-antichymotrypsin (ACT), whereas only about 10% to 30% is present as uncomplexed or free PSA. Circulating antiproteases, such as ACT and alpha 2-macroglobulin (α-2-MG), protect against active proteases. Free PSA also represented an inactive form(s) of PSA, and for this reason was not complexed with protease inhibitors. The inactive, free PSA forms include propSA which is resulted from incomplete cleavage of the pro-sequences during maturation, BPSA which is correlated with benign disease, and in PSA (intact, non-active PSA). Significance of the above isoforms of PSA related to diagnosis of a prostate condition is discussed in more details in Gabriela De Angelis et al., Reviews In Urology, 2007, VOL. 9, page 113-123, Hans Lilja et al., Nature Reviews Cancer, 2008, VOL. 8, page 268-279 and S. D. Mikolajczyk et al., Clinical Biochemistry, 2004, VOL. 37, page 519-528 herein incorporated by reference in their entirety.

In other embodiments, the protease can be one or more of trypsins, chymotrypsins, human kallikreins, matrix metalloproteases (MMP family), cathepsins, which can be found, for example in serum, and additional proteases such as the one described in Mikolajczyk et al Clinical Biochemistry 37 (2004) 519-528 incorporated herein by reference in its entirety and additional proteases identifiable by a skilled person upon reading of the present disclosure.

In some embodiments, methods herein described can be used to multiplex monoparameter assays previously described in connection with detection of protease performed using luciferase and bioluminescence to advance the luciferyl-peptide strategy toward a proteomics tool targeting proteases in a sample and in particular in serum.

The term “monoparameter assay” as used herein refers to an analysis performed to determine the presence, absence, or quantity of one protease. The term “multiparameter assay” refers to an analysis performed to determine the presence, absence, or quantity of a plurality of proteases. The term “multiplex” or “multiplexed” assays refers to an assay in which multiple assays reactions, e.g., simultaneous assays of multiple analytes, are carried out in a single reaction chamber and/or analyzed in a single separation and detection format.

Monoparameter assays that can be performed with the methods herein described include but are not limited to, any assays for the detection of single markers in serum, single protein detection in biological samples, and further assays which are identifiable by a skilled person upon reading of the present disclosure. Many proteases useful for detection are known to those of skill in the art and can be detected and/or captured using the disclosed multi-ligand capture agents and methods.

Multiparameter assays that can be performed with the methods herein described include but are not limited to any proteomic analysis, tissue analysis, serum diagnostics, biomarker, serum profiling, and additional assays identifiable by a person skilled in the art upon reading of the present disclosure.

In some embodiments, wherein methods and systems are used for performing multiparameter assays, the method and systems herein described can comprise contacting a panel of selected substrates with a sample, wherein each of the selected substrates is configured to be cleaved by at least one of multiple proteases of interest. In particular, each of the selected substrates can be specifically cleavable by one or more of the multiple proteases of interest. The method further comprises detecting a bioluminescent signal for each substrate, and the bioluminescent signal pattern that is characteristic for the panel is used as a biomarker for diagnosing a condition. In some of those embodiments, detectably distinguishable labels are used in connection for separate proteases or groups of proteases. The wording “detectably distinguishable” as used herein with reference to labeled molecule indicates molecules that are distinguishable on the basis of the labeling signal provided by the label compound attached to the molecule.

In some embodiments, the contacting can be performed on the multiple proteases of interest pre-separated from the sample.

In some embodiments, the methods and systems herein described can advantageously be used to perform diagnostic assays, wherein the target(s) to be detected are predetermined biomarkers associated to a predetermined condition. The wording “associated to” as used herein with reference to two items indicates a relation between the two items such that the occurrence of a first item is accompanied by the occurrence of the second item, which includes but is not limited to a cause-effect relation and sign/symptoms-disease relation. Exemplary biomarkers include clinically informative biomarkers, and diagnostic biomarkers.

Those embodiments are particularly advantageous in a diagnostic approach where different classes of biomaterials and biomolecules are each measured from a different region of a typically heterogeneous tissue sample, thus introducing unavoidable sources of noise that are hard to quantitate.

Exemplary assays that can be performed with the methods and systems herein described include but are not limited to serum diagnostics, immunohistochemistry and other separations, and enzyme-linked immunosorbent assays.

In some embodiments, methods and systems herein described can be used to diagnose a condition in an individual.

The term “condition” as used herein indicates a physical status of the body of an individual (as a whole or as one or more of its parts), that does not conform to a standard physical status associated to a state of complete physical, mental and social well-being for the individual. Conditions herein described include but are not limited disorders and diseases wherein the term “disorder” indicates a condition of the living individual that is associated to a functional abnormality of the body or of any of its parts, and the term “disease” indicates a condition of the living individual that impairs normal functioning of the body or of any of its parts and is typically manifested by distinguishing signs and symptoms.

The term “individual” as used herein in the context of treatment includes a single biological organism, including but not limited to, animals and in particular higher animals and in particular vertebrates such as mammals and in particular human beings.

In embodiments of methods and systems herein described, the condition is associated to a predetermined concentration of one or more protease in an active form and the one or more active protease is able to specifically cleave a corresponding target peptide. The method comprises contacting a sample from the individual with a substrate herein described comprising the target peptide conjugated with a label, to allow cleavage of the target peptide by the protease. The method further comprises detecting the bioluminescent signal, detecting a concentration of the active protease in the sample based on the detected bioluminescent signal and comparing the detected concentration with the predetermined concentration to diagnose the condition in the individual. The system comprises one or more substrates each comprising the target peptide conjugated with the label and reagents for detecting bioluminescence signal from the label and a look up table comprising predetermined concentrations associated to diagnose of one or more conditions in an individual.

In some embodiments, methods and systems herein described allow accurate detection of active protease (and in particular PSA) among different isoforms, some of which can be a better prognostic indicator of cancer. For examples, some results show that the levels of active PSA in complexed or uncomplexed forms are higher in cancer patients. Since there are no methods of measuring the complexed PSA, measuring the functionality of the uncomplexed PSA should correlate to presence of cancer. Due to the low levels of uncomplexed PSA present in the serum, only an assay such as the one herein described developed can be used to detect the correction.

In particular, in some embodiments the detected ratio of active PSA/total PSA can be detected by detecting the amount of active PSA using method and systems herein described, detecting the amount of total PSA using methods and systems identifiable by a skilled person e.g. including antibody detection targeting for one or more epitopes which are specific for all PSA isoforms or other protein separating/quantification methods known in proteomics, such as ultracentrifugation and chromatography. In some embodiments, when the total PSA is determined by an assay, detection of the total PSA can be performed by detecting active PSA and non-active PSA and determining the total PSA on this basis. In some of those embodiments a margin of mistake can occur based on the sensitivity of the assay used and in some cases may require adjustments and verification assays as will be understood by a skilled person.

In embodiments herein described, amount of PSA or other protease detectable is of about 50 picograms/ml or lower. In embodiments herein described the ratio active PSA/total PSA can be equal or lower than 0.1%. In particular in some embodiments the ratio can be 2 ng active/2000 ng total PSA or 1/1000.

In some embodiments, assays performed according to methods herein described can predict with fewer false positives the diagnosis of prostate cancer in patients exhibiting elevated levels of prostate specific antigen (PSA) in serum

In some embodiments, assays performed according to methods herein described allow a unique approach for diagnosing the probability of carcinoma of the prostate.

In particular, in some embodiments, the ratio of functional activity of PSA present in the serum to the hospital PSA can give a prognosis for patients with elevated PSA levels, with a higher ratio indicating higher probability of cancer

In some embodiments, diagnosis of prostate cancer can in particular be performed by detecting prostate specific antigen activity from serum, comprising using bioluminescence with PSA activable aminoluciferly peptides to determine the ratio of active PSA to total PSA, where in a higher ratio correlates to a higher incidence of prostate cancer.

As disclosed herein, the substrates, labels, and target peptides herein described can be provided as a part of systems to perform any assay, including any of the assays described herein. The systems can be provided in the form of arrays or kits of parts. An array sometimes referred to as a “microarray”, can include any one, two or three dimensional arrangement of addressable regions bearing a particular molecule associated to that region. Usually, the characteristic feature size is micrometers.

In a kit of parts, the substrates, labels, and target peptides and other reagents to perform the assay can be comprised in the kit independently. The capture agent can be included in one or more compositions, and each capture agent can be in a composition together with a suitable vehicle. For example systems herein descried can comprise a support suitable to identify an active protease in a sample. The support comprises a capture agent able to specifically bind the protease in a capture agent-protease binding complex, wherein the protease in the capture agent protease binding complex is proteolytically active. In particular in some embodiments support can be formed by a platform and/or beads which can be incubated with anti-protease capture agents (such as Mab) and used for capturing the protease from the sample (e.g. serum/plasma). The captured protease can then be reacted with the labeled substrate (e.g. aminoluciferin substrate) to release aminoluciferin and quantify the amount of detected protease.

Additional components can include labeled molecules and in particular, labeled polynucleotides, labeled antibodies, labels, microfluidic chip, reference standards, and additional components identifiable by a skilled person upon reading of the present disclosure. In some embodiments, the system for detecting an active protease in a sample can further comprise a cell expressing luciferase as an independent biosensor. In particular, in some embodiments which can in particular be directed to diagnosis of a condition in an individual, a lookup table can be comprised in the system and in particular, the look up table can comprise one or more predetermined concentrations of one or more proteases, wherein the predetermined concentration are associated to diagnosis of one or more conditions in an individual.

The term “lookup table” as used herein indicates a data structure, usually an array or associative array, which can be in physical or computer support and can have various forms as it will be understandable by a skilled person.

In some embodiments, detection of a the protease can be carried either via fluorescent based readouts, in which the labeled antibody is labeled with fluorophore, which includes, but not exhaustively, small molecular dyes, protein chromophores, quantum dots, and gold nanoparticles. Additional techniques are identifiable by a skilled person upon reading of the present disclosure and will not be further discussed in detail.

In particular, the components of the kit can be provided, with suitable instructions and other necessary reagents, in order to perform the methods here described. The kit will normally contain the compositions in separate containers. Instructions, for example written or audio instructions, on paper or electronic support such as tapes or CD-ROMs, for carrying out the assay, will usually be included in the kit. The kit can also contain, depending on the particular method used, other packaged reagents and materials (i.e. wash buffers and the like).

### EXAMPLES

The methods and systems for detecting and profiling proteases and proteases' activity in a sample, related platforms, kits of parts and systems to perform bioluminescent assays and a method and/or system using assays herein disclosed are further illustrated in the following examples, which are provided by way of illustration and are not intended to be limiting.

The following exemplary methods, platforms and system for protease detection and profiling are illustrated in connection with experimental procedures and characterization data performed with reference to PSA.

A person skilled in the art will appreciate the applicability and the necessary modifications to adapt the features described in detail in the present section, to additional proteases, platforms, compositions, methods and systems according to embodiments of the present disclosure.

### Example 1

### Synthesis of Peptide Aminoluciferin Substrates Suitable to be Used as Substrates for Proteases Reactions

Aminoluciferin is treated as an unnatural amino acid that can be introduced into a peptide sequence. Such peptides containing aminoluciferin can then be used as substrates for various proteases. Traditional strategies cannot easily be used to introduce aminoluciferin into a peptide sequence, a synthesis approach has been developed in which higher yields of amino acid/peptide-aminoluciferin conjugation can be achieved. In addition, a new synthetic sequence to produce larger amounts of the necessary starting materials has been developed.

In an exemplary set of experiments, a peptide-conjugated 2-cyano-6-aminoacid-aminobenzothiazole was synthesized according to the following reaction scheme.

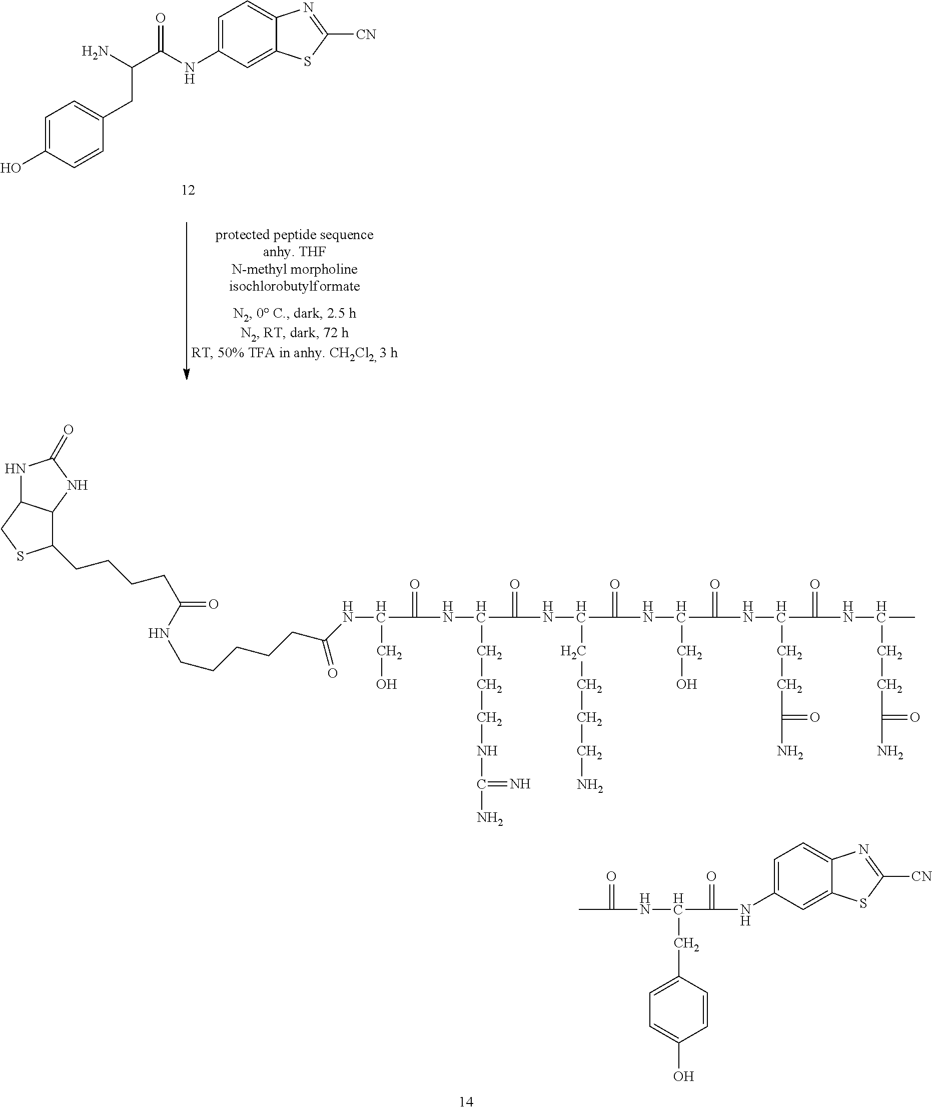

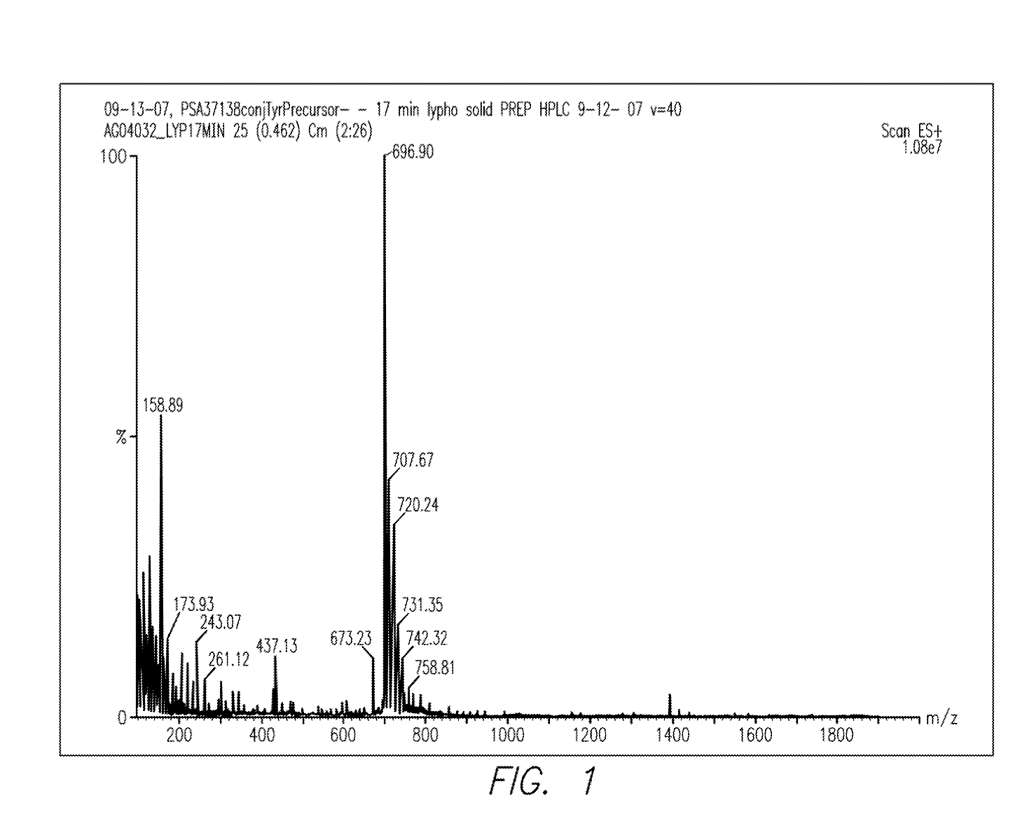

The peptide sequence was designed to be recognized by a specific protease in the blood that is indicative of disease (e.g. prostate specific antigen (PSA), which correlates to prostate cancer; a wide variety of proteases are also present and these levels can change in response to biological insults conferred by infection, malignant growth and autoimmune responses (J Jacobs et al. J Proteome Research, 4, 1073-85 (2005)). The protected peptide sequence (0.033 mmoles, 0.067 g) was dissolved in 2.3 mL of anhydrous THF (+small amount of anhydrous DMF), sonicated, and stirred under N₂ at 0° C. in the dark (material in suspension). N-methylmorpholine (2 eq, 0.07 mmol, 0.007 mL) and isobutyl chloroformate (1.3 eq, 0.04 mmol, 0.006 mL) were added drop-wise to the reaction at 0° C. and stirred for 30 min in the dark. Separately, purified amino acid-conjugated 2-cyano-6-aminobenzothiazole (e.g. tyrosine conjugated) 12 (0.03 mmol, 0.011 g) was dissolved in 0.2 mL of anhydrous THF and then added dropwise to the reaction flask over a period of 30 min. The reaction was stirred under N₂ in the dark at 0° C. for 2 h, followed by a 72 h stir at room temperature in the dark. After 72 h, the anhydrous THF/DMF was removed in vacuo. The product 14 was dissolved in EtOAc and washed with saturated NaHCO₃ to quench any remaining isobutyl chloroformate. The organic layer was collected and evaporated to dryness to yield a bright yellow to golden orange/yellow residue. The protected group on the peptide portion of the product was removed using 50% TFA in anhydrous CH₂Cl₂; stirred at RT in the dark for 3 h. Following deprotection, the product 14 was evaporated to dryness and placed on vacuum. Compound 14 was identified with LC/MS spectroscopic analysis as illustrated in FIG. 1.

This same procedure can be applied for various other peptide sequences as a means to develop new conjugates as bioluminescence probes for detection of various other proteases that link to disease.

The peptide-conjugated tyrosine-D-aminoluciferin was synthesized according to the following reaction scheme.

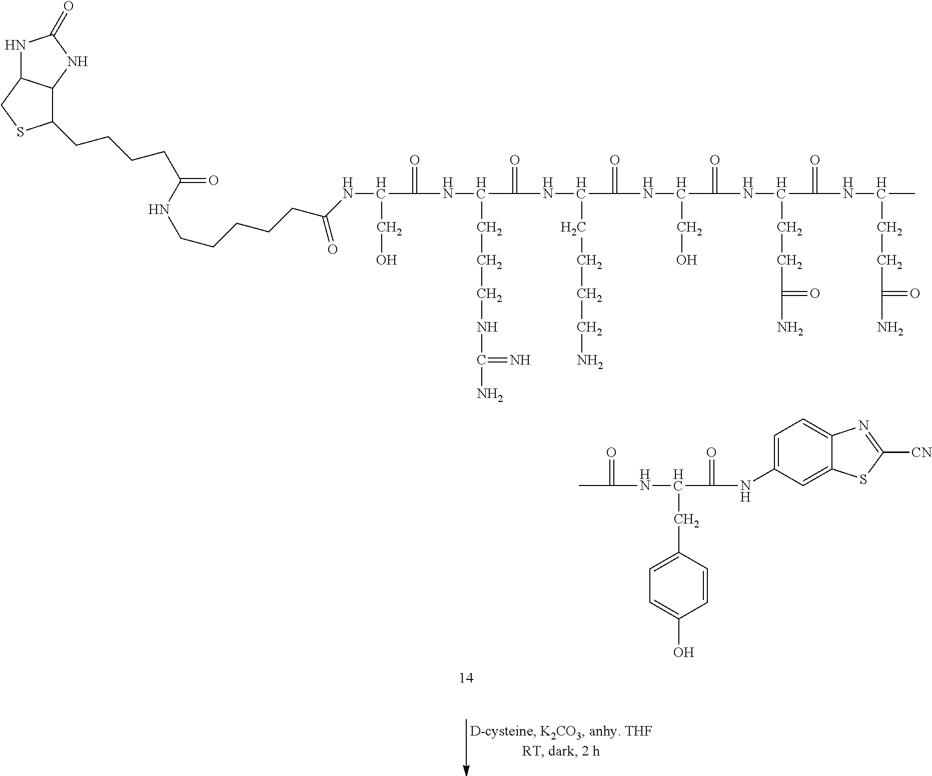

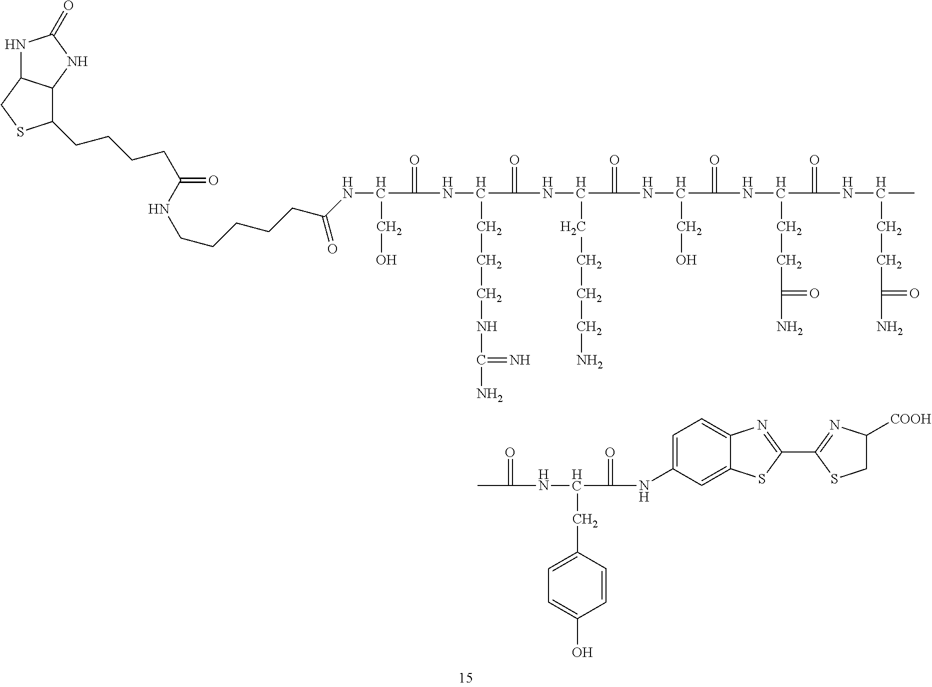

The deprotected and purified peptide-conjugated 2-cyano-6-aminobenzothiazole-[amino acid] (0.006 mmoles, 0.008 g) was dissolved in anhydrous THF (0.01 mL), followed by drop-wise addition of D-cysteine (1.2 eq, 0.007 mmoles, 0.001 g, 0.001 mL) while stirring the reaction at RT in the dark for 2 h. After 2 h, the reaction was evaporated to dryness and placed on vacuum. The residue was re-dissolved in a mixture of anhy. THF and any solid residue removed by filtration through a 0.45 μm filter. The THF was removed in vacuo.

### Example 2

### Synthesis and Screening Methods to Identify Suitable Protease Substrates

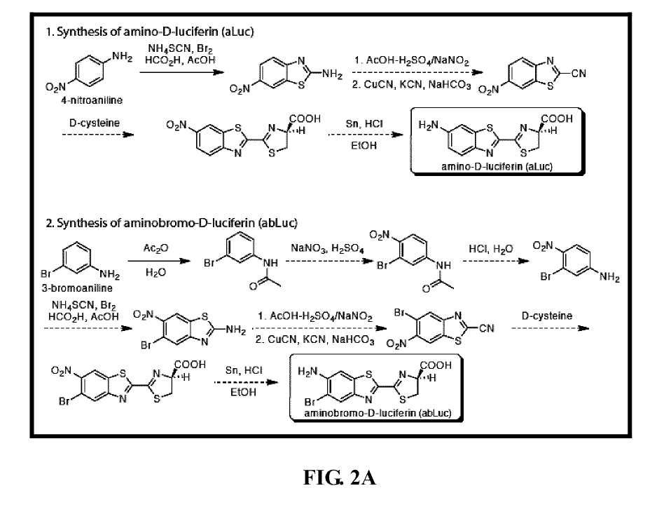

Combinatorial chemistry is expected to be suitable for synthesis and screening to identify optimal substrates for PSA as illustrated in FIG. 2.

In particular, a multigram scale synthesis of luciferin analogs can be prepared following synthetic schemes such as the ones illustrated in FIG. 2A wherein natural D-luciferin, amino-D-luciferin (aLuc) and aminobromo-D-luciferin (abLuc) analogs are prepared and incorporated in a one-bead-one-compound library as indicated. In particular, these luciferin analogs are expected to be suitable in a solid-phase peptide combinatorial library by split-mix approach as schematically shown in FIG. 2B. In the approach illustrated in FIG. 2B, modified luciferin analogs can be conjugated to peptides by combinatorial method to produce over 100,000 luciferyl peptidic conjugates on beads.

The beads with the conjugates resulting from the above approach can then be screened against PSA according to the approach illustrated in FIG. 2C. In particular, to identify targeting peptides against these markers, the beads will be screened against the PSA, and the substrate efficacy will then be assessed by addition of luciferase. After the initial screening, identified luciferyl peptidic substrates will be submitted for structural identification by microsequencer and MALDI-TOF.

### Example 3

### PSA Activity Assay

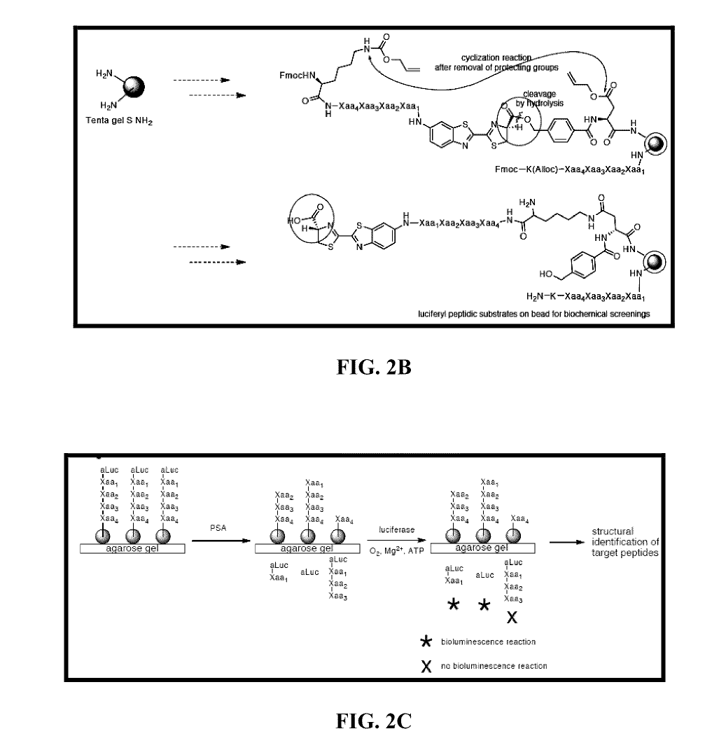

PSA activity and related concentration in a sample can be tested using a bioluminescent label according to the reaction scheme schematically illustrated in FIG. 3. FIG. 3 shows a scheme depicting an exemplary application of bioluminescent assay performed with labeled probes obtainable with the methods and systems herein described.

In particular, in the illustration of FIG. 3, a peptide substrate when interacting with a specifically designed protease probe has the capability to release the D-aminoluciferin, which ultimately provides light output in the presence of Mg²⁺ and ATP when proteases (e.g. indicative of disease) are present in the biological sample. In some embodiments, this assay can serve as a marker for disease

Currently used assay for PSA activity is fluorescence-based with a sensitivity of 0.5 ng/ml. However, most specific substrates for PSA (HSSKLQ-aluc and SKLQ-aluc) do not have high enzyme turnover. Use of bioluminescent assay increases detection limit significantly.

Aminoluciferyl substrates are 1000 times more sensitive than methylcoumarin based substrates and 100 times more sensitive than based rhodamine based substrates for caspase detection (O'Brien et al., J. Brien Biomol Biomol. Screening, 10 10, 137 , 137-48 (2005)).

An assay was performed to test detection PSA activity in buffer using bioluminescence. Specifically, PSA at known concentrations from 0.001 to 100 ng/ml in volume of 50 μl was incubated with SKLQ-aluc substrate in a Tris A buffer for 14 hours. After the incubation, 50 μl of luciferin detection reagent (LDR) [40 mM Tris-acetate, 1 mM EDTA, 1 mM DTT, 3.45 mM ATP, 0.2 M NaCl, 5.7 mM MgSO₄, and 0.76 mM coenzyme A pH (7.6)] was added to 75 μl of the reaction mixture according to the manufacture's (Promega) instruction. Bioluminescent signal produced by the reaction between LDR and free aluc was registered in a Berthold luminometer with a delay of 1 second.

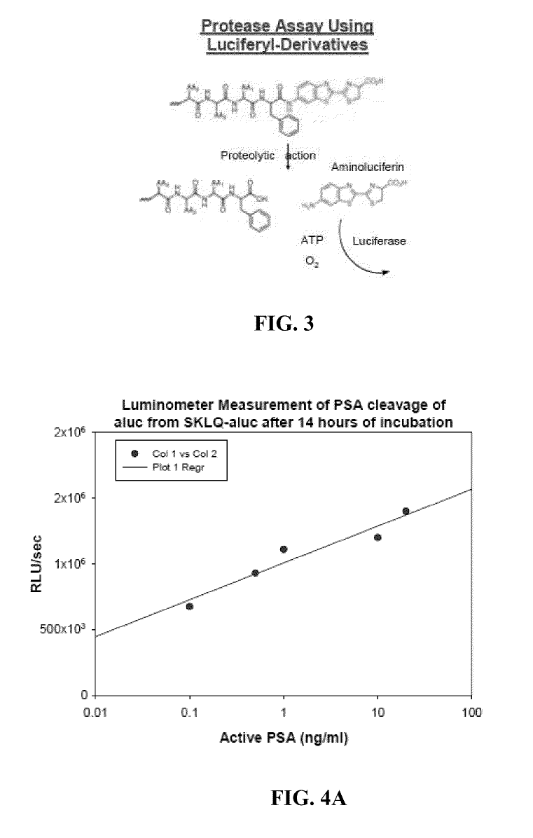

The Berthold luminometer outputs the rate of photon emission by bioluminescence reactions in photons per second (RLU/sec). Photon emission is proportional to the amount of produce (e.g. oxyluciferin) formed in a bioluminescent reaction. The photon emission rate was multiplied by the time period between measurements, to give the total photons emitted in each period. Addition of these photons collected in each time period yielded a cumulative profile of the bioluminescence product formed over the course of a bioluminescent reaction (total RLU). FIG. 4A shows the measurement of the rate of photon emission (RLU/sec) as a function of PSA concentration (ng/ml). A measurement of the net rate of photon emission (RLU/sec) associated to PSA concentration (ng/ml) is generated by subtracting the background/noise signal from FIG. 4A, and the result is shown in FIG. 4B.

### Example 4

### Development of Reactions Conditions for PSA Detection

In order to identify reactions conditions suitable to obtain PSA detection, release of aLuc from aminoluciferyl-peptides upon cleavage by a protease and luciferase-aminoluciferin reaction were measured in various reaction buffers and compared in order to find the optimum buffer for the particular protease-substrate pair.

In particular, different buffers were screened for optimum functioning of both enzymes, PSA and luciferase. This screen allows one to select buffers that provide the highest substrate cleavage by PSA and then the highest bioluminescence signal of this free aluc by luciferase according to the experimental design. In particular, the screen was directed to select a buffer that could be used for the entire assay.

In particular, a first Buffer PSA-A comprising 50 mM Tris-HCL and 0.15 mM NaCl was tested and compared with a second buffer PSA-B that comprised PSA-A (50 mM Tris-HCL and 1.5 mM NaCl)

The results are summarized in Tables 1A and 1B.

TABLE 1A
Luciferase-aminoluciferin reaction

| Buffer | RLU/s |
|---|---|
| HEPES | 509510 |
| PBS | 2114628 |
| PSA-A (0.15M NaCl) | 6884224 |
| PSA-B (1.5M NaCl) | 24042 |
| Tris-Acetate | 1245484 |
| Gly-gly | 14053778 |
| Water | 47 |

TABLE 1B
PSA release of aluc in different buffers as measured by luciferase

| Buffer | RLU/s |
|---|---|
| HEPES | 2043 |
| PBS | 1285448 |
| PSA-A (0.15M NaCl) | 4678533 |
| PSA-B (1.5M NaCl) | 19149 |
| Tris-Acetate | 1820828 |
| Gly-gly | 13556333 |
| Water | 97 |

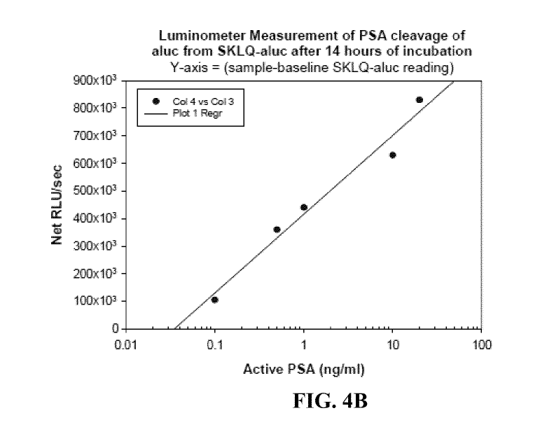

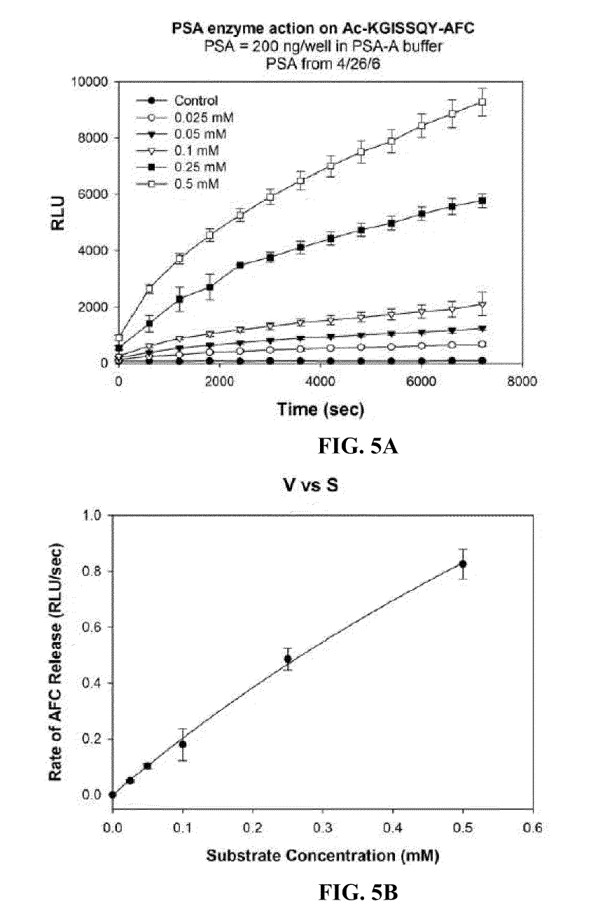

The kinetics of the reactions performed with the two buffers was also tested as illustrated in FIGS. 5 and 6.

FIG. 5A and FIG. 6A show representative example of the RLU for PSA with a fluorogenic substrate Ac-KGISSQY-AFC. The slope of these curves under steady-state conditions gave the rate of product formation, v, at different initial substrate concentrations. FIG. 5B and FIG. 6B show the rate of product formed, v, with an increasing substrate concentration, S. The Kₘ values indicate the highest affinity and Vₘₐₓ values show the highest rate of photon emission. The catalytic efficiency of the enzymatic reaction is obtained by the ratio of Vₘₐₓ/Kₘ.

Specifically, a first penal of 6 reactions is performed in a multi-well microtiterplate. In each well, 200 ng of PSA is mixed with a fluorogenic substrate Ac-KGISSQY-AFC of certain concentrations (0 mM, 0.025 mM, 0.05 mM, 0.1 mM, 0.25 mM and 0.5 mM) in PSA-A buffer of Example 4. First, the RLU measured by a luminometer at specific time point of each reaction is plotted as a function of reaction time (FIG. 5A), and the _{Km} value is estimated to be 1.73 mM. Second, the rate of AFC release v, is plotted against substrate concentration, S (FIG. 5B), and the Vₘₐₓ is estimated to be 3.7 RLU/sec.

Similarly, a second penal of 6 reactions is performed in a multi-well microtiterplate. In each well, 100 ng of PSA is mixed with a fluorogenic substrate Ac-KGISSQY-AFC of certain concentrations (0 mM, 0.1 mM, 0.25 mM, 0.5 mM, 1 mM and 2 mM) in PSA-B buffer of Example 4. First, the RLU measured by a luminometer at specific time point of each reaction is plotted as a function of reaction time (FIG. 6A), and the Kₘ value is estimated to be 0.47 mM. Second, the rate of AFC release v, is plotted against substrate concentration, S (FIG. 6B), and the Vₘₐₓ is estimated to be 2.33 RLU/sec.

Based on the above estimations, the catalytic efficiency of the PSA. Ac-KGISSQY-AFC reaction is much higher in the PSA-A buffer as compared to in the PSA-B buffer, which is consistent with the results presented in Table 1.

### Example 5

### Development of a Platform for Performing the Protease Activity Assay

The protease activity assay can be performed on various platforms, which include but does not limit to a platform suitable for a bead-based protease activity assay, microplate-based protease activity assay, or a protease activity assay in free solution.

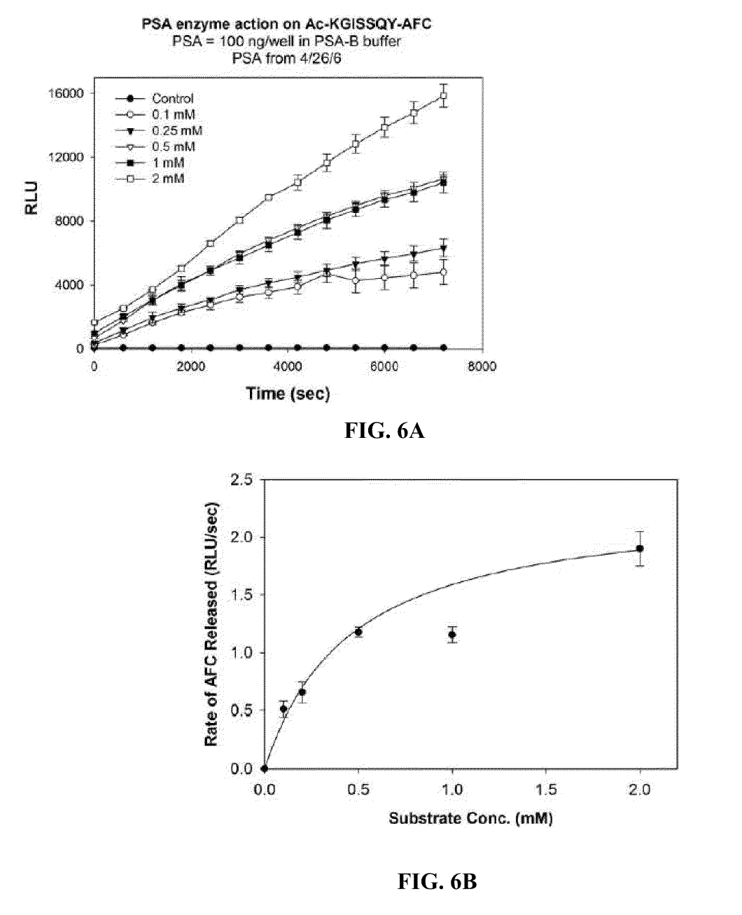

For example, FIG. 7A shows a bead-based PSA activity assay, wherein the luciferase-aminoluciferin reaction is carried out in a reaction tube containing anti-PSA antibody coated Protein A agarose beads and aminoluciferyl-peptide substrate for PSA. As described in Example 6 below, the anti-PSA antibody coated beads can be used to immunocapture PSA from a serum sample added to the tube, and a semi-preamble membrane (cutoff filter) is used to reduce background signals in the assay. Further, a variety of beads can be used in the platform described above, which include but not limit to, magnetic monoclonal coated-beads and other monoclonal coated-polymer such as sepharose. This procedure can be used for various purposes and in particular for research purposes and detection performed on a single substrate. Techniques suitable to manufacture beads such as the one exemplified in FIG. 7A by attaching the antibodies or other capture agents to a suitable support in the sense of the present disclosure will be identifiable by a skilled person upon reading of the present disclosure.

Another example of the platform is provided by FIG. 7B showing a microplate-based PSA activity assay, wherein the luciferase-aminoluciferin reaction is carried out in one or more wells of a microtiterplate, which is coated with a layer of anti-PSA antibody coated protein A. As described in Example 6 below, each well can contain a aminoluciferyl-peptide designed and synthesized directed to a specific class of protease, and multiple serum samples can be added to each well individually to obtain a functional profile of protease activity in the sample. This platform can be used in particular for clinical applications related to detection of both single PSA and proteomics. Techniques suitable to manufacture arrays attaching the antibodies or other capture agents to various supports to form a platform in the sense of the present disclosure will be identifiable by a skilled person upon reading of the present disclosure.

### Example 6

### Selection of Antibodies for Platforms Suitable for PSA Detection

Selection of proper antibodies has been performed to enable pre-selection and immobilization of PSA with antibody while minimizing the interference of the antibody with the PSA active site and consequence impairment of the enzymatic activity.

In particular, experiments were done to determine if a Mab blocks the active site of PSA and to select the Mab that were not blocking the active site according to the desired effect and experimental design.

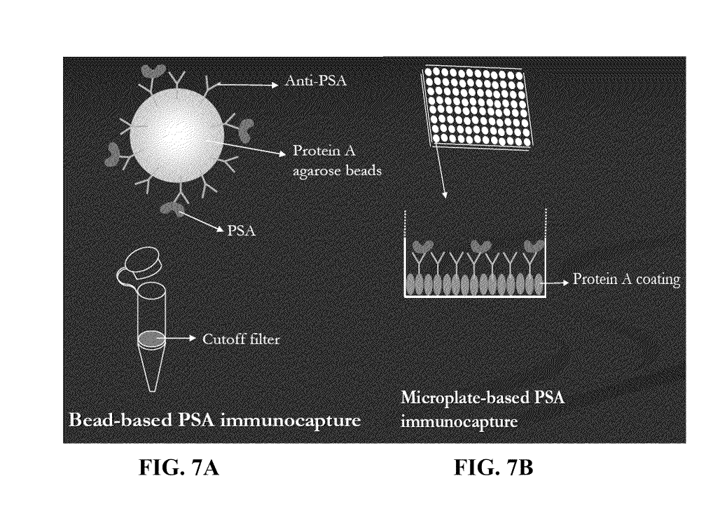

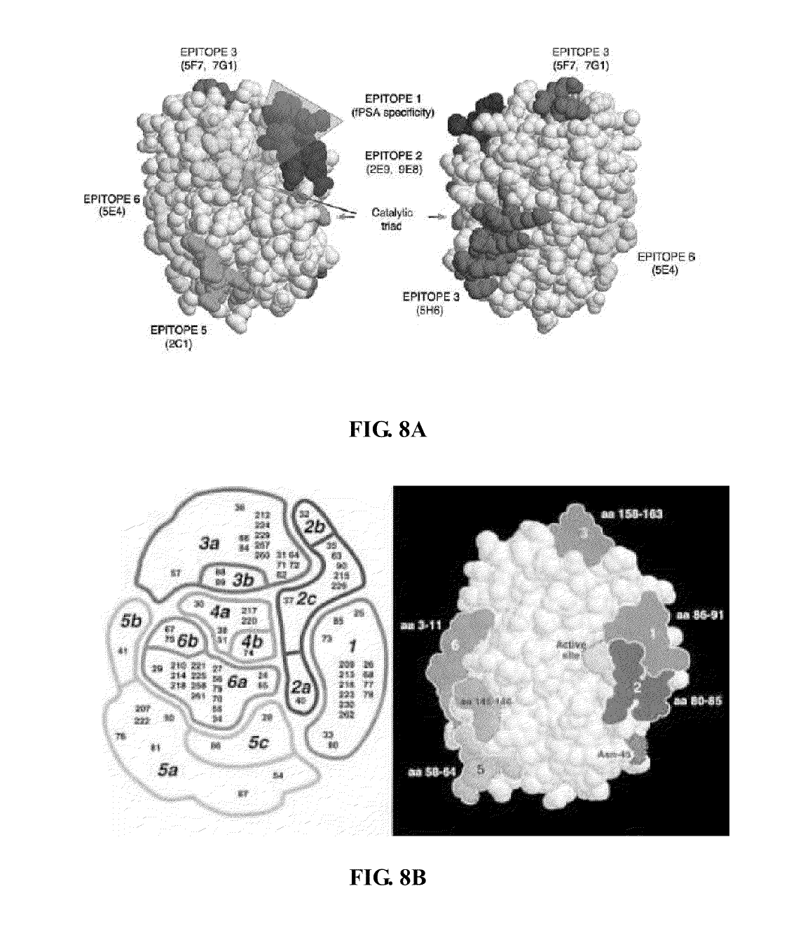

In particular, Applicants performed a selection of antibody in view of the structure and location of related epitopes schematically illustrated in FIG. 8

Monoclonal Antibodies for PSA were therefore purchased from US Biologicals, in particular two antibodies Mab26 and Mab30 were selected that target epitope4 (IgG1) and 6 (IgG2a) on PSA. Selection was also performed in view of lack of cross reactivity with hK2 or other homologous proteins.

Experiments were performed to test ability of the Mabs with fluorogenic Substrate for PSA Ac-KGISSQY-AFC wherein a 3 fold excess of the Mab was used to initiate the blocking. After incubation, the PSA was incubated with a fluorescence substrate and the enzymatic activity as measured by AFC released was quantified.

In particular, to determine whether the two antibodies Mab 26 and Mab 30 block the active site of PSA, 5 μg PSA was incubated with 0.5 mM of the fluorogenic substrate for PSA Ac-KGISSQY-AFC alone (PSA), or together with 15 μg (3-fold in amount) of Mab 30 (PSA/Mab-30) or Mab 26 (PSA/Mab-26). In a negative control, no PSA was added to the substrate (blank).

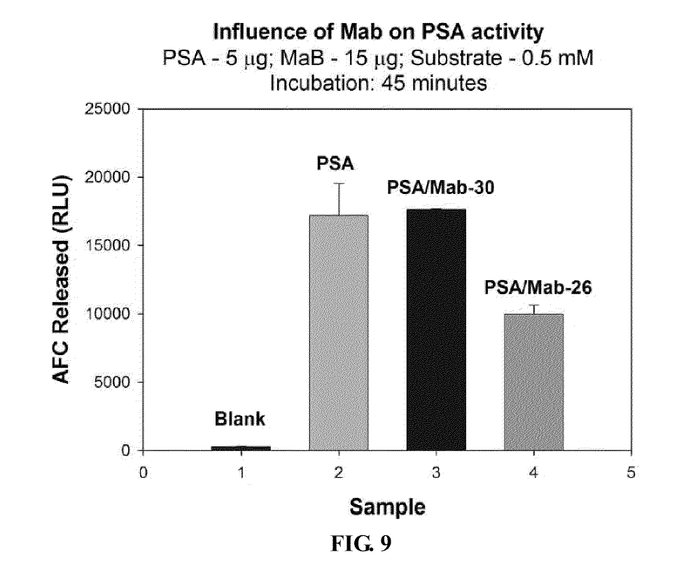

The results are illustrated in FIG. 9. The negative control shows little enzymatic activity of PSA. Noticeably, PSA/Mab-30 exhibits similar PSA activity as compared to the reaction with PSA alone, and an obviously higher PSA activity as compared to PSA/Mab-26. This result indicates that Mab-26 has a greater effect in blocking the active site of PSA as compared to Mab-30, which appears to show reduced blocking of enzymatic site

### Example 7

### Antibody Based Assays to Detect Proteases

The antibodies identified in Example 6 were used in assays to detect PSA activity.

In a first set of the experiments, a Bead-Based Assay has been performed with the Mabs of Example 6. Coat Protein-A beads with the anti-PSA antibody were provided. Abundant proteins from serum were removed and the serum was incubated with beads coated with anti-PSA antibody and subsequently washed. The beads were incubated with PSA substrate the beads were filtered and washed. The substrate was collected with free aminoluciferin. The luciferase activity was then detected.

In a second set of experiments, a microplate-based assay was performed. In particular, anti-PSA antibodies were incubated in Protein-A coated wells. The serum was cleared off abundant proteins such as HSA and IgG. The sample was applied to the well and incubated to capture PSA. PSA was then applied, and unattached proteins washed off. The wells were incubated with the PSA substrate and the supernatant substrate solution was measured using luciferase in a luminometer.

According to both procedures the microtiter plate containing aminoluciferyl-peptides and serum samples were incubated in the PSA-A buffer of Example 4 for 14 hours to allow PSA in the serum samples to cleave the aminoluciferin off of the peptide.

After the incubation a semi permeable membrane is placed over the reaction. The semi-permeable membrane would allow only aminoluciferin to move across the barrier and get oxidized by the luciferase to emit light, thus reducing noise in the assay. After filtration by the semi permeable membrane, a luciferin detection reagent (LDR) cocktail consisting of luciferase (1 ng/μl), ATP (3.45 mM), MgSO₄ (5.7 mM), DTT (1 mM), NaCl (0.2 M) and Co enzyme A (0.76 mM) is added to detect and measure the bioluminescent signal generated from the assay. All the assays were carried out in triplicate, and the bioluminescent signal was measured over 15 minutes using a IVIS 200 device.

Further, in the microplate-based assay, each well of the microplate can contain a specific sequence that is recognized by one, or a class of, proteases. For example, in one assay, each well of the microplate can contain an aminoluciferyl peptide of a different sequence, the different aminoluciferyl-peptide sequences are designed to be recognized and cleaved by PSA at an proteolytic efficiency characteristic for the sequence. For the PSA assay, the peptide sequence can be for example anyone of KGISSQY (SEQ ID NO: 1), SRKSQQY (SEQ ID NO: 2), GQKGQHY (SEQ ID NO: 3), EHSSKLQ (SEQ ID NO: 4), QNKISYQ (SEQ ID NO: 5), ENKISYQ (SEQ ID NO: 6), ATKSKQH (SEQ ID NO: 7), KGLSSQC (SEQ ID NO: 8), LGGSQQL (SEQ ID NO: 9), QNKGHYQ (SEQ ID NO: 10), TEERQLH (SEQ ID NO: 11), GSFSIQH (SEQ ID NO: 12), HSSKLQ (SEQ ID NO: 13), SKLQ (SEQ ID NO: 14), KLQ (SEQ ID NO: 15), LQ (SEQ ID NO: 16). A functional profile of PSA in the serum sample can thus be obtained using the bioluminescent signal generated by the released aminoluciferin in the assay

Alternatively, the different aminoluciferyl-peptide sequences can be designed to be recognized and cleaved by multiple proteases that are potentially present in the serum sample, so that a functional profile of different proteases in the serum sample can be obtained through the assay. Examples of the multiple proteases to be detected in the assay include but are not limited to, trypsins, chymotrypsins, human kallikreins, matrix metalloproteases (MMP family), cathepsins.

A functional profile of different proteases in the serum samples can therefore be obtained through the bioluminescent signal detected from the assay. Those results can be compared to a functional profile of a serum sample from a normal healthy patient (see Example 12 below). Further, the functional profile of a combination of proteases can be used to diagnose or obtain a prognosis of a disease afflicting the patient (see Example 12).

Since the protease activity assay herein disclosed based on detection of bioluminescence is more sensitive than any of the state-of-the art techniques including those use fluorescence or mass spectrometry data, very small amounts of proteases, ideally at pg/ml levels, can be screened using this assay.

### Example 8

### Immunocapture and Detection of Active PSA-Antibodies Concentrations and Reaction Conditions

The antibody selected as exemplified in Example 6 were used in assays to detect PSA activity according to procedures exemplified in Example 7 with various concentrations of antibody to verify the impact of antibody concentrations on the ability to capture active PSA included in a sample.

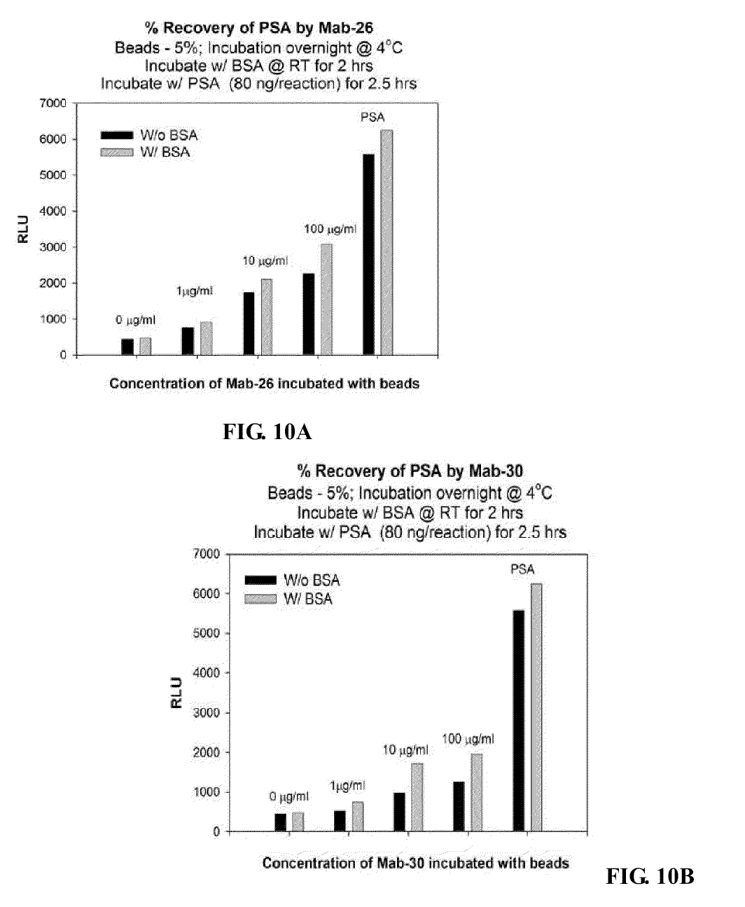

In particular, Mab 30 and Mab26 were tested to verify how efficiently one can immobilize the PSA in the pre-purification step from solution using this beads and how much PSA activity is retained after such immobilization. The results are shown in FIG. 10.

Specifically, Mab-26 or Mab-30 was added to 5% protein A coated beads to reach a final concentration of the Mab of 1, 10 or 100 μg/ml. The mixture was then incubated at 4° C. overnight to allow associate of the Mab to the beads. After the incubation, BSA was added to the beads and incubated for 2 hours at the room temperature to allow BSA to block hydrophobic sites on the beads. Then the beads were incubated with 80 ng of PSA with constant agitation for 2.5 hours to allow immunocapture of PSA from the mixture.

Finally, the beads were recovered from the mixture and washed with a Tris buffer for three times to remove free PSA and incubated with 0.5 mM of Ac-KGISSQY-AFC c substrate to allow PSA enzymatic activity. In a negative control group, no Mab was incubated with the beads. In a positive control group, 80 ng of PSA was directly added to react 0.5 mM of Ac-KGISSQY-AFC substrate.

The results illustrated in FIG. 10 show no substantial difference in the active PSA detected with both Mabs due to BSA presence. Further, Mab-26 exhibit a higher efficiency in immunocapture of PSA as compared to Mab-30

### Example 9

### Immunocapture and Detection of Active PSA-Concentrations

The antibody selected as exemplified in Example 6 were used in assays to detect PSA activity according to procedures exemplified in Examples 7 and 8 with various initial PSA concentrations to verify the impact of initial PSA concentrations on the ability of the antibody to capture active PSA included in a sample.

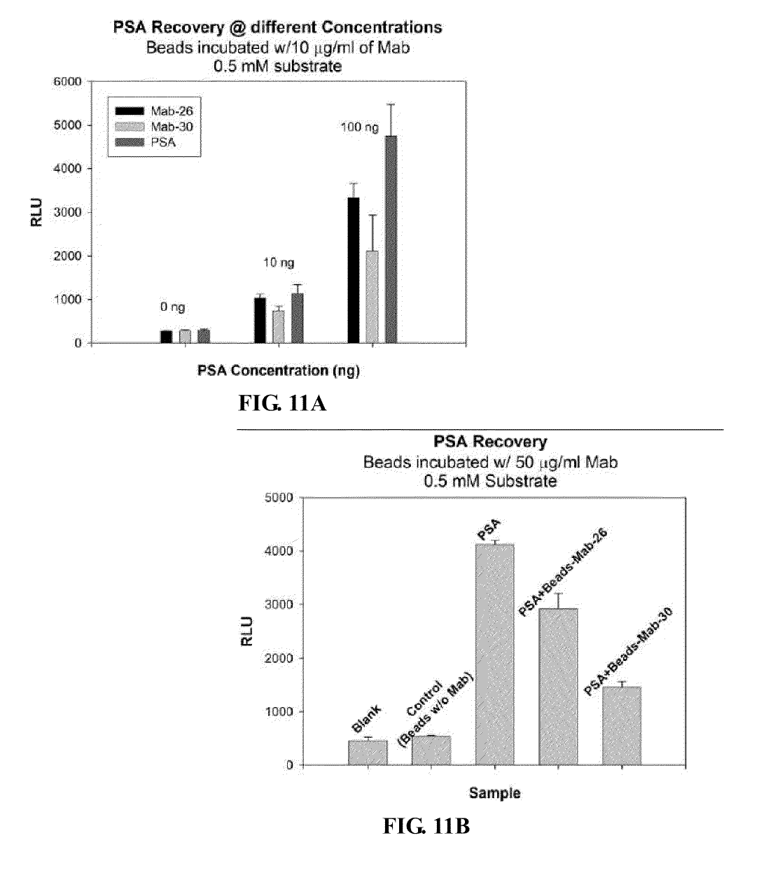

A first set of experiments was performed to test PSA recovery at different initial PSA concentrations. The results are shown in FIG. 11 A. Specifically, Mab-26 or Mab-30 was added to 5% protein A coated beads to reach a final concentration of the Mab of 10 μg/ml. The mixture was then incubated at 4° C. overnight to allow associate of the Mab to the beads. After the incubation, the beads were incubated with 10 or 100 ng PSA with constant agitation for 2.5 hours to allow immunocapture of PSA from the mixture. Finally, the beads were recovered from the mixture and washed with a Tris buffer for three times to remove free PSA and incubated with 0.5 mM of Ac-KGISSQY-AFC substrate to allow PSA enzymatic activity. In a negative control group, no Mab was incubated with the beads. In a positive control group, 10 or 100 ng PSA was directly added to react with 0.5 mM of Ac-KGISSQY-AFC.

A second set of experiments was performed to compare Mab 26 and Mab 30 in terms of PSA recovery by the PSA activity. The results are shown in FIG. 11B. Specifically, Mab-26 or Mab-30 was added to 5% protein A coated beads to reach a final concentration of the Mab of 50 μg/ml. The mixture was then incubated at 4° C. overnight to allow associate of the Mab to the beads. After the incubation, the beads were incubated with 100 ng PSA with constant agitation for 2.5 hours to allow immunocapture of PSA from the mixture. Finally, the beads were recovered from the mixture and washed with a Tris buffer for three times to remove free PSA and incubated with 0.5 mM of Ac-KGISSQY-AFC substrate to allow PSA enzymatic activity. In a first negative control group (blank), no PSA was added to the beads. In a second negative control group (control), no Mab was added to the beads. In a positive control group (PSA), 100 ng PSA was directly added to react with 0.5 mM of Ac-KGISSQY-AFC.

The results illustrated in FIG. 11 show a better performance by Mab 26 with respect to the one of MAb-30. In this connection, even if MAB 26 appeared inferior to Mab 30 under the immunocapture experiments illustrated in Example 6 and FIG. 9 in term of exposing the PSA active site the experiments illustrated in the present example indicated that Mab 26 is more efficient than Mab30 in term of recovery by the activity. Therefore it appears that there is an impact on the efficiency of the test due to the ability on an antibody's side to recover the protease.

Taken together, the results of Examples 6-9 indicate that an antibody that hinders the active site can still perform well because of a better ability to recover the protease in active form (compare FIG. 9 and FIG. 11B). Also the concentrations of antibody used as well as the initial PSA concentration in a sample both have an impact on recovery of active PSA from the sample (see FIGS. 10 and 11)). Further, BSA does not affect the recovery of active PSA using the Mabs (see FIG. 10).

In general, several factors could impact the assay. For example, the epitope to which an antibody binds can block completely or partially the active site of a protease of interest. The experimental condition e.g. buffer, incubation time and/or temperature are also expected to have an impact, on the tertiary structure (folding/unfolding) of the enzyme and therefore can on the positioning of the antibody with respect to the protease active site. Also, the incubation time of the enzymatic reactions can also affect a final outcome of a protease activity assay such as the one herein described. In some instances, the bioluminescent signal as well as a background signal can be dependent on the amount of protease present in a sample, the amount of antibody used to purify the protease of interest, and the amount of detecting reagent (e.g. luciferase) used. Finally, the concentration of Mab on beads can also impact the efficiency of recovery of active PSA from a sample.

Accordingly, using different buffers can change the folding or opening up of PSA and luciferase as will be understood by a skilled person. The incubation times of PSA with substrate and then luciferase with substrate can also influence the final result as will be understood by a skilled person. For example, the amount of PSA can be fixed for a given patent per ml of blood and a higher or lower bioluminescence signal and/or background signal, can be obtained based on the amount of luciferase uses in the detection. The concentration of Mab on beads can also impact the efficiency of PSA removal from blood

One skilled in the art will understand the opportunity to perform measurements to take into account impact of concentrations, location of the epitope of an antibody with respect to the active site of the protease and ability on the antibody's side to recover protease in active form. In determining the effective active amount to be used and the effective amount to be retrieved all the factors should be taken into account according to the experimental design.

### Example 10

### Immunocapture and Antibody Studies of PSA-Target Peptide

The antibody selected as exemplified in Example 6 were used in assays to detect PSA activity according to procedures exemplified in Example 7 with various target peptides.

In particular in a first set of experiments the two target peptides for PSA KGISSQY and SKLQ are tested in human serum.

In particular, Human Plasma (female) was incubated with 4 ml of 10% bead solution and with 50 μg/ml of Mab-26 overnight. 500 and 1000 ng/ml of purified active PSA were mixed in 4 ml of plasma and incubated for 5 hours. 600 μl of beads per sample were placed into 6 centrifuge tubes w/0.2 μm filter, wash 2-times to remove excess Mab. 1.45 ml of plasma was added to each tube and mixed for 2.25 hours. Plasma was filtered and washed 2-times. 400 μl of substrate (0.5 mM of Ac-KGISSQY-AFC) or (0.15 mM of SKLQ-Aluc) was added to retentate beads in a solution which has 1 mg/ml BSA. The resulting mixture was incubated for 22 hours. For aluc, 100 μl of sample+50 μl of LDR was used per reaction.

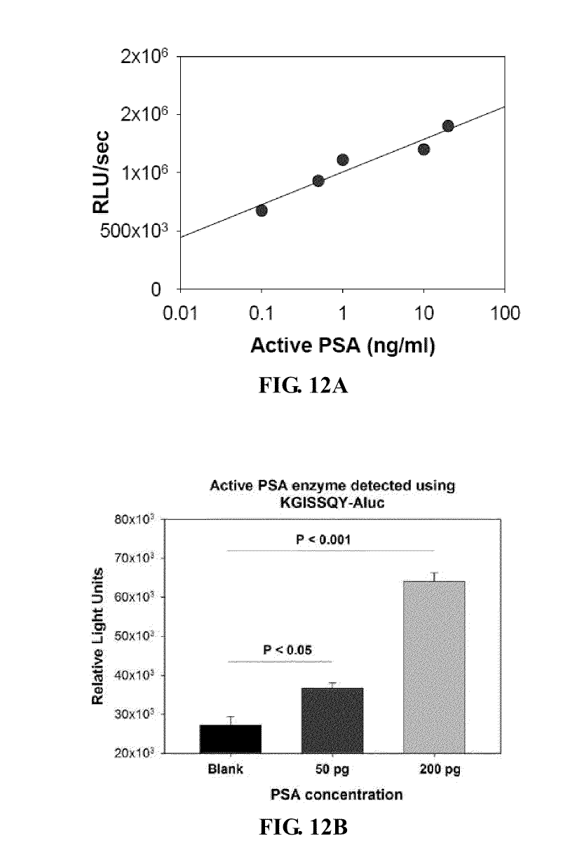

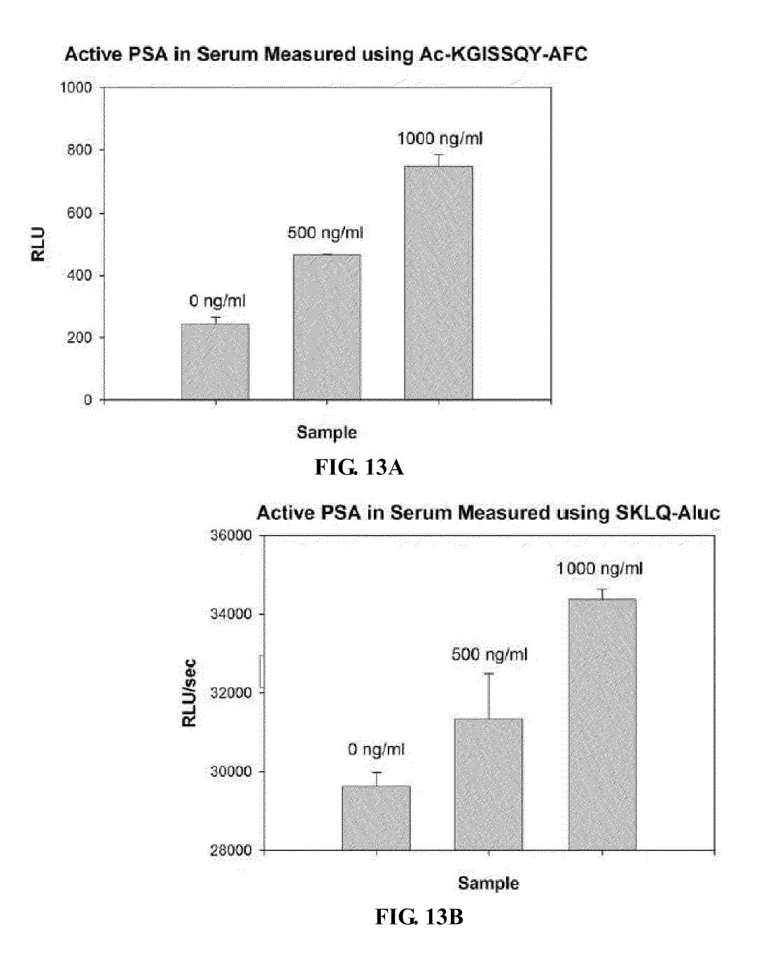

The results illustrated in FIG. 12 and FIG. 13 show that the substrate of KGISSQY has a much higher turnover rate by PSA as compared to SKLQ. Thus, in view of the results obtained with experiments illustrated in the present examples, a skilled person will understand that KGISSQY has a very rapid turnover by PSA, but also has low specificity (e.g. it can it can also be recognized and cleaved by other serum proteases such as chymotrypsin of human) compared to SKQL. SKLQ on the other had appears to have a very slow turnover compared to KGISSQY and to be highly specific to PSA.

In this connection, the results illustrated in FIG. 12 and FIG. 13 also suggest that even though the PSA turnover rate for SKLQ is relatively low, trace amount of active PSA of as low as 0.1 pmole/ml present in a serum sample can still be detected, due to the ultra sensitivity of the bioluminescent reaction. Typically, the bioluminescent assay is 100-1000 fold more sensitive than fluorescence-based assays.

Therefore, even at (x-fold) lower enzymatic turnover of SKLQ by PSA, one can detect bioluminescence using luciferase. Using a KGISSQY-aluc sequence for bioluminescence, one can instead enhance the sensitivity of the assay many fold. Bioluminescence is 100-1000 fold more sensitive that fluorescence, especially since there is no background signal in a bioluminescence reaction.

### Example 11

### Optimization of the Protease Activity Assay

Protease substrate peptide sequence. For a single, or a class of, protease(s) to be tested and/or detected with the protease activity assay, a rational design of the substrate peptide sequence is performed according to known sequences recognized and cleaved by the protease(s). For example, for a PSA activity assay, 14 PSA substrate peptides of 4-7 amino acid sequences (Seq ID NO. 1-14) are synthesized using a peptide synthesis method known in the art. When selection of an optimal substrate peptide is necessary, tests of PSA activity and specificity on these substrates as well as other properties, for example stability of the substrates in sera, can be performed according to the methods of S. R. Denmeade et al. (Cancer research, 1997, vol. 57, page 4924-4930).

Optimum buffer for the assay. Speed of release of aLuc from aminoluciferyl-peptides upon cleavage by a protease and speed of luciferase-aminoluciferin reaction in various reaction buffers is measured and compared in order to find the optimum buffer for the particular protease-substrate pair (see Example 4 above).

Reduced background and enhanced sensitivity. Some amino acids that are conjugated to aminoluciferin can also act as substrates for luciferase and thus generate a higher background signal in the assay than other amino acid. Assay. Therefore, the substrate peptides directed to specific classes of protease are designed and synthesized such that the amino acids adjacent to the aminoluciferin are those associate with lower background in the assay. The aminoluciferyl-peptides with reduced background can be used alone or together with the semi-permeable membrane filtration as in Example 4 to reduce background signals of the protease activity assay.

The protease activity assay can be further improved with higher specificity by employing antibodies to isolate the target protease before assaying its functionality. The advantage of pre-immunocapture of the protease using an antibody is that even small amounts of proteases can be detected, which can otherwise escape detection by other modalities.

Suitable antibody for immunocapture of a protease from a serum sample. Optimization of the antibody for a certain protease such as PSA can be identified based on information on the positioning of epitopes and active site in the protease known in the art or retrievable with methods herein described and with additional method also identifiable to a skilled person. For example in case of PSA information on the active site and structure of the protease can be found also in view of the information described in Huhtinen et al., J. Immuno. Methods, 294, 111-122 (2004) and in Tumor Biology-Workshop, 20, 1-12 (1999) each incorporated herein by reference in its entirety.

### Example 12

### Detection of Proteases for Diagnostic Purposes

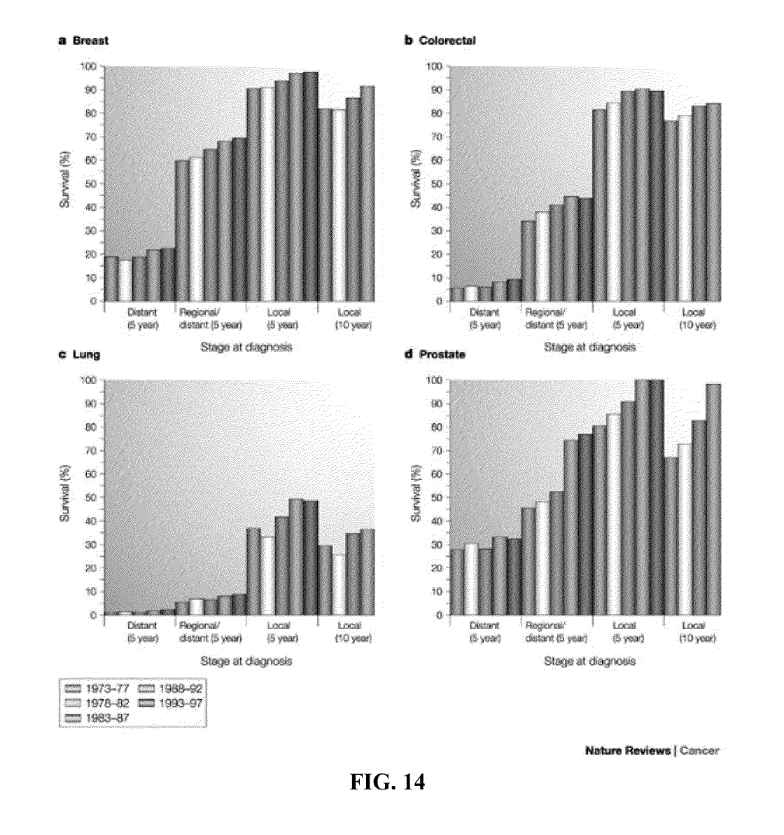

It has been established that early detection typically improves the possibility to localize and potentially cure diseases. Early disease detection and assessment is therefore expected to dramatically effect therapeutic outcome (see FIG. 14)

Biofluids (serum/plasma, urine, saliva etc.) do not contain genome or transcriptome data. Since they interface with tissues, these fluids can hold information pertaining to disease states. Proteases are part of the serum proteome and are associated to pathologic conditions (cancer, inflammation, infection and cardiovascular disease). Serum protease profiling using sensitive assays could potentially provide diagnostic and prognostic data. Since bioluminescence does not require excitation, and has a high quantum yield compared to fluorescence and other techniques, it is possible to detect extremely low levels of active proteases. Applicants are exploiting this sensitivity to probe low levels of active proteases in serum and correlate them with disease state.

TABLE 2
Projected changes in survival with early detection

| | | | 5-year survival rate |
|---|---|---|---|
| | | | if all tumors were |
| | Tumors localized | 5-year survival | localized when |
| Cancer Site | when detected (%) | rate (%) | detected (%) |
| Colorectal | 41 | 64 | 90 |
| Lung | 19 | 16 | 49 |
| Breast | 65 | 87 | 97 |
| Prostate | 65 | 90 | 100 |

Based on data from SEER¹ for cases diagnosed between 1900 and 1999 inclusive. Cases with in situ or unstaged disease have been excluded. The favorite overall 5-year survival among breast and prostate cancer patients is partly due to the prevalence of screening for these cancers during the calendar years considered.

Serum contains several proteases that could individually or in combination with other proteases and/or other biomarkers diagnose or provide prognosis of a number of different diseases. Applicants have previously described protease assays using aminoluciferin as a reporter but here take the next step in multiplexing these assays to advance the luciferyl-peptide strategy toward a proteomics tool targeting serum proteases. These assays are based on the use aminoluciferin as iii substrate for firefly luciferase treated as an unnatural amino acid that can be introduced into a peptide sequence.

Such peptides containing aminoluciferin can then be used as substrates for various proteases. Upon cleavage of the peptide by the target protease the aminoluciferin will then be released and act as a substrate for luciferase enzyme. Since traditional strategies cannot easily be used to introduce aminoluciferin into a peptide sequence, a new synthesis approach has been developed in which higher yields of amino acid/peptide-aminoluciferin conjugation can be achieved. In addition. a new synthetic sequence to produce larger amounts of the necessary starting materials has been developed. Some amine acids conjugated to aminoluciferin act as substrates for luciferase and generate a higher background signal than others, In this approach, we use a rational design directed at specific classes of proteases and incorporate amino acids adjacent to the aminoluciferin that reduce the background signal and specifically target the proteases with a set of aminoluciferin-peptides.

The assay involves synthesis of different aminoluciferyil-peptide sequences that are then added to multi-channel microtiter plates ranging from 6 to 1536 unique elements or greater. Each well contains a specific peptide and serum samples from patients are added and microtiter plates are incubated such that the proteases in the serum samples cleave the luciferin off of the peptide. Since each of the wells contains a specific sequence that is recognized by one, or a class of, proteases, the aminoluciferin should be cleaved off if the target protease(s) are present in the serum sample. After the Incubation of the serum with the different peptides a semi permeable membrane is placed over the reaction and a cocktail consisting of luciferase, ATP, MgSO₄, DTT, NaCl and Co enzyme A is added to each well to measure the bioluminescent signal generated, A semi permeable membrane would allow only aminoluciferin to move across the barrier and get oxidized by the enzyme to emit light , thus reducing noise in the assay (see Example 5). As aminoluciferyl peptides with reduced background are developed (see Example 12), it is possible to eliminate the membrane in these assays and add the reaction buffer directly to the cleaved substrate.

A functional profile of different proteases in the sera can thus be obtained using the bioluminescent signal generated by the released aminoluciferin, The results can be compared to the functional profile of a serum sample from a normal healthy patient. Hence, these functional profiles of a combination of proteases can be used to diagnose or obtain a prognosis of the disease afflicting a patient. Since bioluminescence is more sensitive than any of the state of the art techniques that use fluorescence or mass spectrometry data, very small amounts of proteases can be screened using this assay. Abnormal proteolytic activities of one or multiple enzymes can provide significant data to obtain trends or biomarkers for numerous diseases. This assay does not require the use of antibodies to separate target proteases, and is comparable or even more sensitive than ELISA. Also, due to the sensitivity of the assay, small amounts of serum samples may be needed. The assay can be made even more specific by employing antibodies to isolate the target protein before assaying its functionality. The advantage of using an antibody is that even small amounts of proteases can be detected, which can otherwise escape detection by other modalities.

Proteolytic enzymes are expected to be present in extremely low quantities that can be important indicators of the disease state , which have not been identified by existing assay methods. In order to reduce further any background signal from the aminoluciferyl-peptides, it is also possible to test a luciferase expressing cellular setup that can be added to each of the wells, thus providing an independent biosensor approach that uses the strength of living cell sensors that can generate the detecting luciferase enzyme, ATP and MgS0₄.

### Example 13

### Detection of PSA for Diagnostic Purposes

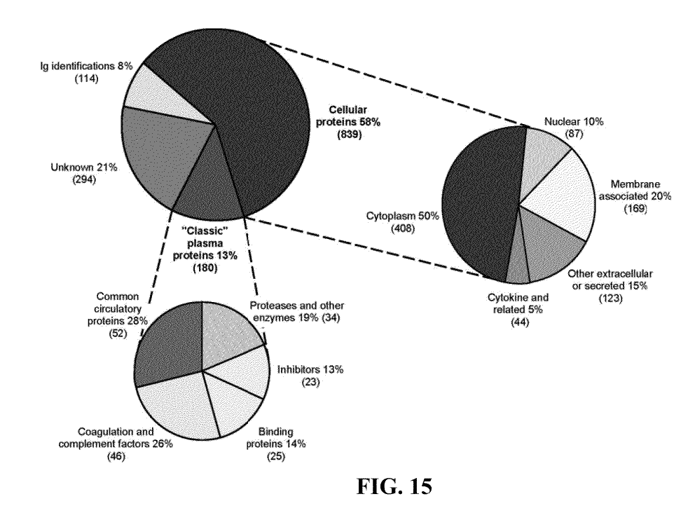

Plasma proteins is formed by any of the various dissolved proteins of blood plasma, including antibodies and blood-clotting proteins, that act by holding fluid in blood vessels by osmosis. FIG. 15 shows a schematic representation of the categorization of plasma protein from J. Jacobs et al., J. Proteome Research, 4, 1073-85 (2005) incorporated herein by reference in its entirety. Only 18% of these proteins are formed by proteases.

Depending on the protease selected there might be a trade-off for higher enzyme turnover (>10 times) versus greater specificity in the selection procedure. Therefore, depending on the protease to be targeted previous immunocapture is expected to increase the specificity towards similar proteins which have a greater turnover (see e.g. PSA v. chymotrypsin) (see Example 10).

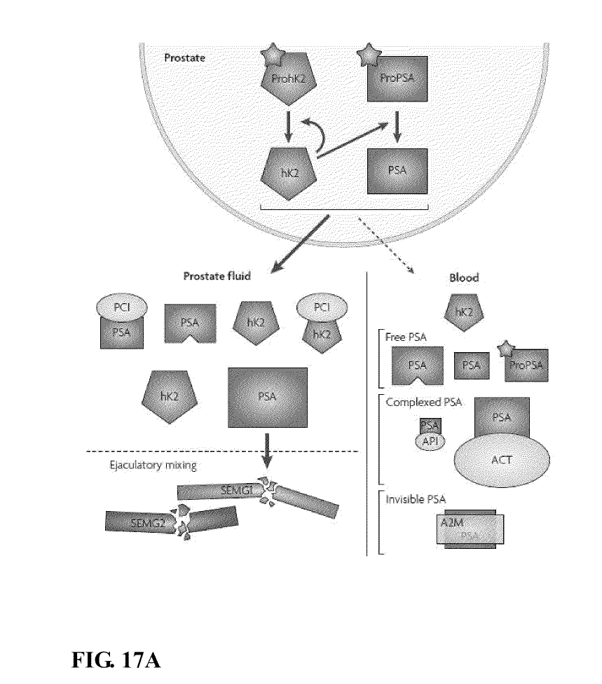

FIG. 17 shows a schematic representation of the various PSA isoforms and the respective relevance as biomarker. In particular, FIG. 17A shows prostate-specific antigen (PSA) subforms and interactions. Active forms of PSA and kallikrein-related peptidase 2 (hk2) are shown in red, inactive forms in blue or green. In the prostate, propeptides (grey wedge) are removed from propSA and prohK2, leaving the mature, catalytic forms. PSA forms in prostatic fluid are active PSA, nicked PSA (niPSA) and PSA complexed with protein C inhibitor (PCI). The size in the figure indicates the relative abundances of the forms. Blood contains a variety of forms of PSA: free PSA forms (nicked, intact and propSA) and complexed PSA. The most abundant form in blood is PSA complexed with 1-protease inhibitor (API) are estimated to comprise only a 1-2% or lower proportion of PSA in blood. A2M envelopes PSA, masking the epitopes recognized by commercial PSA assays and thus rendering this form invisible to the assays.

FIG. 17B shows forms of free PSA in serum. The free PSA in serum is composed of three major forms: pro-PSA, BPSA, and in PSA. Only the percentage of pro-PSA is elevated in cancer, while BPSA and in PSA are associated to benign diseases.

FIG. 17C shows association of free PSA forms with prostate cancer. Overall the percentage of free PSA (free PSA/total PSA) is decreased in cancer. The % in PSA (in PSA/free PSA) component of free PSA is decreased in cancer. BPSA is associated to BPH, which can coexist with cancer. BPSA is generally lower in prostate cancer though not a strong diagnostic marker for the presence of cancer. The % pro-PSA (pro-PSA/free PSA) is the only component of free PSA that increases with the presence of cancer.

Recent work on whether ratios of free PSA or PSA/total PSA is a better marker for cancer·Target various forms of PSA using antibodies·Total PSA levels in blood>4 ng/ml is correlated to greater cancer risk·PSA secretion is elevated in cancers, benign prostatic hyperplasia (BPH) and also increases with age.

TABLE 3
Accuracy of % pPSA to differentiate PCa from BPH in total PSA range
of 2-10 ng/ml

| Total PSA (ng/ml) | Sensitivity | Unnecessary Biopsies Spared |
|---|---|---|
| 2-4 | 90% | 19% |
| 4-10 | 90% | 31% |

In the conventional clinical diagnostics, the probability of a patient having prostate cancer is determined by quantifying the biomarker PSA levels in serum. The PSA is typically measured by ELISA. When PSA concentration is greater than 4 ng/ml, the patient is asked to undergo further tests, such as biopsies to determine whether, the elevated SA levels are cause by cancer. Recent clinical data has shown that the amount of total SA in serum is not a very good indicator of prostate cancer.

PSA levels in blood are increased in the case of inflammation, benign prostatic hyperplasia (BPH) or with age. PSA is a secreted protein that is expressed in a pre-pro-form. The precursor of the pro-PSA is transported across the membrane and released into the extracellular space, where it is activated. The transmembrane portion of the protein has 24 amino acids. The proform of PSA has 7 amino acids that need to be cleaved to activate the PSA. This activation step is thought to be carried out by among other trypsin-like proteases, human kallikrein (hK2), which is also secreted by prostate tissue. More recently, it has been observed that PSA exists in various isoforms. The PSA activation step can lead to incomplete cleavage of the 7 amino acid chain, thus providing inactive PSA with 2, 4, or 5 amino acids, also known as free PSA. hK2 is elevated in cancer as a compared to BPH and higher levels of Hk2 can yield more active forms of PSA. Active PSA when released into the serum forms complexes with &#61537: antichymotrypsin (ACT), &#61537: —antitrypsin (AT), and a host of other serine protease inhibitors. Free PSA is not known to form complexes with any of the inhibitors. Attempts have been made to correlate the free-PSA levels to cancer. Some studies suggest that a higher concentration of free-PSA correlate to lower incidence of cancer. These various isoforms of PSA are identified using monoclonal antibodies targeted towards different epitopes on the PSA molecule, each of which is found to present the different isoforms.

Detection of active PSA by these immunoassays can be challenging in particular if directed to obtain functional information of PSA due to either lower sensitivity of the technique or lack of good monoclonal antibodies targeted towards the active site of PSA. Most of the active PSA exist in a complexed form, with ACT or AT. Some reports suggest that 1-3% of the total PSA is active and uncomplexed. These low levels (1-3% of 4 ng/ml) of PSA are very difficult to detect by current immunoassays. Bioluminescence is significantly more sensitive than fluorescence and Applicants' results show that low picogram levels of active PSA that can be found in serum of patients with elevated PSA levels. There have been attempts to correlate prostate cancer prognosis to the various isoforms of PSA. For example a higher ratio (−2) PSA to total PSA was found to correlate to lower rates of cancer with the negative ratio indicating that the detected PSA is formed entirely by an inactive isoform of PSA, due for example to incomplete cleavage of the 7 aa pre-sequence of pro-PSA during maturation. Some work has also been attempted to correlate the ratio of complexed PSA-ACT/total PSA to cancer, with higher ratio indicating a greater probability of cancer. No one has been able to correlate the ratio of active PSA/total PSA to the incidence of cancer due to the above mentioned difficulties. Using bioluminescence with PSA actable aminoluciferyl peptides, a higher ratio of active PSA/total PSA is expected by Applicant to correlate to a higher incidence of prostate cancer according to the present disclosure.

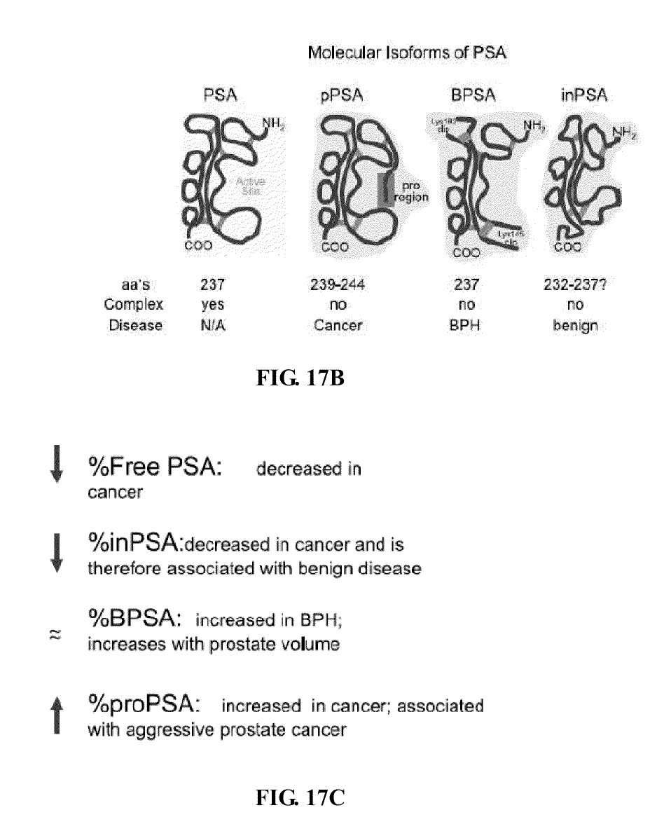

An assay to test the ratio and/or other features associated to active proteases can be performed according to the schematics of FIG. 18. In particular, the assay can be developed on different platforms, e.g. magnetic monoclonal coated-beads, monoclonal coated-polymer such as sepharose, coated microtiterplate or in free solution (see Example 5). The serum to be diagnosed can be used directly by addition of a PSA specific aminoluciferyl peptide and sufficient incubation. Later, a luciferase cocktail containing known amount of luciferase, MgSO₄, ATP, NaCl, DTT, EDTA and Co enzyme-A is added to the serum-aminoluciferyl peptide reaction mixture. Upon appropriate incubation, the active PSA cleaves aminoluciferin from the peptide which is the quantified using the luciferase cocktail. In another approach, the total PSA can be immunopurified from the serum using appropriate antibodies. The antibodies to be used are targeted towards the total PSA, such that the PSA activate site is left readily accessible for the aminoluciferyl peptide. Only apportion of the total PSA that is captured is active aminoluciferyl peptide. Only a portion of the PSA that is captured is active. In order to immunocapture the PSA antibodies can be attached to beads, polymer support or in microtiter plates. Their binding efficacy can be enhanced by using a linker or other antibodies such as streptavidin. Once the PSA is immunocaptured, a series of wash steps are applied to remove other entities present in the serum. In this manner, a pure PSA population present in the serum can be captured for further assays. To this captured PSA, the aminoluciferyl peptides are applied and incubated for the appropriate time. After the incubation, during which sufficient amounts of the aminoluciferin is released from peptide, the luciferase cocktail is added and bioluminescence is measured. If the aminoluciferyl peptides exhibit a significant background, as can be the case for certain aminoluciferyl sequences, then the incubation mixture can be applied to a membrane such that the bigger peptide or uncleaved peptide is retained whereas aminoluciferin is collected as the filtrate and mixed with the luciferase-cocktail and quantified. In another novel approach, we also plan to use a luciferin regenerating enzyme (LRE) to recycle oxyluciferin the product of the aminoluciferin thus providing a long lasting signal. The LRE can be added to the luciferase cocktail for optimum and longer lasting bioluminescence signal.

This PSA assay developed by Applicants is extremely sensitive and can detect the functionality of low amounts of PSA. The ratio of functional activity of PSA present in the serum to the hospital PSA can give a prognosis for patients with elevated PSA levels, with a higher ratio indicating higher probability of cancer.

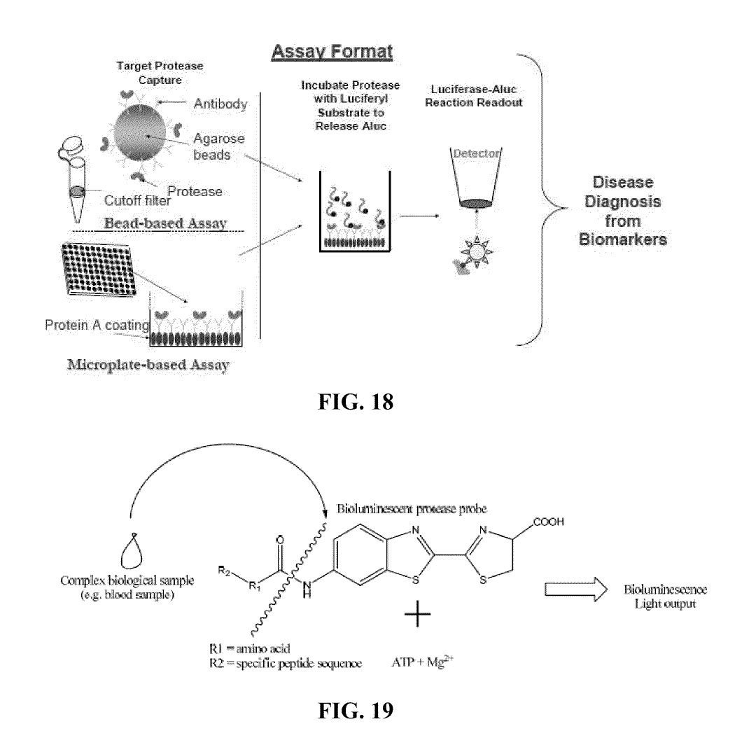

Additional peptides and labels can also be used to perform the PSA assay exemplified in the present section, as well other bioluminescent assays according to the present disclosure. FIG. 19 shows a cartoon scheme depicting an exemplary application of bioluminescent assay performed with labeled probes obtainable with the methods and systems herein described. In particular, in the illustration of, a complex biological sample (ex. blood sample) when interacting with a specifically designed protease probe has the capability to release the D-aminoluciferin, which ultimately provides light output in the presence of Mg2+and ATP when proteases (e.g. indicative of disease) are present in the biological sample. In some embodiments, this assay can serve to identify biomarkers for a condition and in particular a disease.

The examples set forth above are provided to give those of ordinary skill in the art a complete disclosure and description of how to make and use the embodiments of the arrangements, devices, compositions, systems and methods of the disclosure, and are not intended to limit the scope of what the inventors regard as their disclosure. All patents and publications mentioned in the specification are indicative of the levels of skill of those skilled in the art to which the disclosure pertains.

The entire disclosure of each document cited (including patents, patent applications, journal articles, abstracts, laboratory manuals, books, or other disclosures) in the Background, Summary, Detailed Description, and Examples is hereby incorporated herein by reference. All references cited in this disclosure are incorporated by reference to the same extent as if each reference had been incorporated by reference in its entirety individually. However, if any inconsistency arises between a cited reference and the present disclosure, the present disclosure takes precedence. Further, the hard copy of the sequence listing submitted herewith and the corresponding computer readable form are both incorporated herein by reference in their entireties.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the disclosure has been specifically disclosed by preferred embodiments, exemplary embodiments and optional features, modification and variation of the concepts herein disclosed can be resorted to by those skilled in the art, and that such modifications and variations are considered to be within the scope of this disclosure as defined by the appended claims.

It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

When a Markush group or other grouping is used herein, all individual members of the group and all combinations and possible subcombinations of the group are intended to be individually included in the disclosure. Every combination of components or materials described or exemplified herein can be used to practice the disclosure, unless otherwise stated. One of ordinary skill in the art will appreciate that methods, device elements, and materials other than those specifically exemplified can be employed in the practice of the disclosure without resort to undue experimentation. All art-known functional equivalents, of any such methods, device elements, and materials are intended to be included in this disclosure. Whenever a range is given in the specification, for example, a temperature range, a frequency range, a time range, or a composition range, all intermediate ranges and all subranges, as well as, all individual values included in the ranges given are intended to be included in the disclosure. Any one or more individual members of a range or group disclosed herein can be excluded from a claim of this disclosure. The disclosure illustratively described herein suitably can be practiced in the absence of any element or elements, limitation or limitations which is not specifically disclosed herein.

A number of embodiments of the disclosure have been described. The specific embodiments provided herein are examples of useful embodiments of the disclosure and it will be apparent to one skilled in the art that the disclosure can be carried out using a large number of variations of the devices, device components, methods steps set forth in the present description. As will be obvious to one of skill in the art, methods and devices useful for the present methods can include a large number of optional composition and processing elements and steps.

In particular, it will be understood that various modifications may be made without departing from the spirit and scope of the present disclosure. Accordingly, other embodiments are within the scope of the following claims.

What is claimed is:

1. A method to diagnose a condition in an individual, the condition being associated to a predetermined concentration of one or more active proteases, the one or more active proteases being able to specifically cleave a corresponding target peptide, the method comprising
contacting a sample from the individual with a substrate comprising the target peptide conjugated with a label, the substrate configured to allow release of the label upon cleavage of the target peptide by the protease, the label configured to produce a bioluminescent signal upon release from the substrate, the contacting being performed for a time and under condition to allow cleavage of the target peptide by the protease;
detecting the bioluminescent signal,
detecting a concentration of the active protease in the sample based on the detected bioluminescent signal, and
comparing the detected concentration with the predetermined concentration to diagnose the condition in the individual.

2. The method of claim 1, wherein the detected concentration is ≦about 1 ng.

3. The method of claim 1, wherein the detected concentration is ≦about 1 pg.

4. The method of claim 1, wherein the detected concentration is ≦about 1 fg.

5. The method of claim 1, wherein the detected concentration is ≦about 1 ag.

6. The method of claim 1, wherein the protease comprises a plurality of isoforms and active protease is at least one or the plurality of isoforms.

7. The method of claim 1, wherein the condition is prostate cancer and the protease is PSA.

8. The method of claim 7, wherein the predetermined concentration of PSA in an active form is 50 pg/ml.

9. A system to diagnose a condition in an individual, the condition being associated to a predetermined concentration of one or more active proteases, the one or more active proteases being able to specifically cleave a corresponding target peptide the system comprising
one or more substrates each comprising a target peptide conjugated with a label, the substrate configured to allow release of the label upon cleavage of the target peptide by the protease, the label configured to produce a bioluminescent signal upon release from the substrate;
reagents for detecting bioluminescence signal from the label; and
a look up table comprising predetermined concentrations of the one or more proteases associated to diagnose of one or more conditions in an individual.

10. The system of claim 9, wherein the substrate is an aminoluciferyl peptide, wherein the reagent for detecting the bioluminescence signal comprises luciferase.

11. The system of claim 9, further comprise a luciferin regenerating enzyme (LRE), wherein the luciferin is capable of regenerating enzyme recycles oxyluciferin into aminoluciferin.

12. The system of claim 9, wherein the substrate further comprises at least two of one or more amino acids with each amino acid having a side chain hydrophobic, hydrophilic, acid or basic in nature.

13. The system of claim 12, wherein the substrate comprises a tyrosine residue.

14. The system of claim 12, wherein the reagents for detecting bioluminescence signal is in the form of biological cells, wherein the cells naturally or are engineered to produce one or more molecules that facilitate the detecting of bioluminescence signal from the label.

15. A method to diagnose a prostate condition in an individual, the condition associated to prostate-specific antigen (PSA) in the individual, the PSA comprising an active PSA and an inactive PSA, the method comprising
detecting a concentration of a total PSA in a sample from the individual, the total PSA comprising the active PSA and the inactive PSA;
detecting a concentration of the active PSA in the sample;
determining a ratio active PSA/total PSA based on the detected concentration of total PSA and the detected concentration of active PSA and
comparing the determined ratio to a predetermined ratio associated to the prostate condition in the individual, wherein the predetermined ratio is ≦about 0.1%.

16. The method of claim 15, wherein detecting a concentration of the active PSA in the sample is performed by providing a substrate comprising a target peptide conjugated with a label, the target peptide being specifically cleavable by the active PSA, the substrate configured to allow release of the label upon cleavage of the target peptide by the protease, the label configured to produce a bioluminescent signal upon release from the substrate;
contacting the sample with the substrate for a time and under condition to allow cleavage of the target peptide by the PSA;
detecting the bioluminescent signal; and
detecting the active PSA in the sample based on the detected bioluminescent signal.

17. The method of claim 15, wherein the detected active PSA is present in the sample in a concentration ≦about 1 ng.

18. The method of claim 15, wherein the detected active PSA is present in the sample in a concentration ≦about 1 pg.

19. The method of claim 10, wherein the prostate condition is prostate cancer.

20. A method to diagnose a condition in an individual, the condition being associated to a predetermined concentration of a plurality of active proteases, each protease of the plurality of active proteases being able to specifically cleave a corresponding target peptide, the method comprising
contacting a sample from the individual simultaneously with a plurality of substrates each comprising the corresponding target peptide conjugated with a label, the substrate configured to allow release of the label upon cleavage of the corresponding target peptide by the active protease able to specifically cleave the corresponding target, the label configured to produce a bioluminescent signal upon release of the bioluminescent label from the substrate, the contacting being performed for a time and under condition to allow cleavage of the target peptide by the active protease;
detecting the bioluminescent signal,
detecting a concentration of each of the plurality of active proteases in the sample based on the detected bioluminescent signal, and
comparing the detected concentrations with the predetermined concentration to diagnose the condition in the individual.

21. The method of claim 20, wherein the plurality of proteases comprising trypsins, chymotrypsins, human kallikreins, matrix metalloproteases (MMP family), cathepsins and other proteases known in the art.

22. A system to diagnose a condition in an individual, the condition being associated to a predetermined concentration of plurality of active proteases, each protease of the plurality of active proteases being able to specifically cleave a corresponding target peptide, the system comprising
one or more substrates each comprising the target peptide conjugated with a label, the substrate configured to allow release of the label upon cleavage of the target peptide by the protease, the label configured to produce a bioluminescent signal upon release from the substrate;
reagents for detecting bioluminescence signal from the label; and
a look up table comprising predetermined concentrations of the one or more proteases associated to diagnose of one or more conditions in an individual.

23. The system of claim 22, wherein the label comprises detectably distinguishable label, each conjugated to a target specific for a protease of the plurality of proteases.


SEQUENCE LISTING (16 sequences)

SEQ ID NO: 1 - protein, 7 aa, artificial sequence
  Synthetic polypeptide
KGISSQY

SEQ ID NO: 2 - protein, 7 aa, artificial sequence
  Synthetic polypeptide
SRKSQQY

SEQ ID NO: 3 - protein, 7 aa, artificial sequence
  Synthetic polypeptide
GQKGQHY

SEQ ID NO: 4 - protein, 7 aa, artificial sequence
  Synthetic polypeptide
EHSSKLQ

SEQ ID NO: 5 - protein, 7 aa, artificial sequence
  Synthetic polypeptide
QNKISYQ

SEQ ID NO: 6 - protein, 7 aa, artificial sequence
  Synthetic polypeptide
ENKISYQ

SEQ ID NO: 7 - protein, 7 aa, artificial sequence
  Synthetic polypeptide
ATKSKQH

SEQ ID NO: 8 - protein, 7 aa, artificial sequence
  Synthetic polypeptide
KGLSSQC

SEQ ID NO: 9 - protein, 7 aa, artificial sequence
  Synthetic polypeptide
LGGSQQL

SEQ ID NO: 10 - protein, 7 aa, artificial sequence
  Synthetic polypeptide
QNKGHYQ

SEQ ID NO: 11 - protein, 7 aa, artificial sequence
  Synthetic polypeptide
TEERQLH

SEQ ID NO: 12 - protein, 7 aa, artificial sequence
  Synthetic polypeptide
GSFSIQH

SEQ ID NO: 13 - protein, 6 aa, artificial sequence
  Synthetic polypeptide
HSSKLQ

SEQ ID NO: 14 - protein, 4 aa, artificial sequence
  Synthetic polypeptide
SKLQ

SEQ ID NO: 15 - protein, 3 aa, artificial sequence
  Synthetic polypeptide
KLQ

SEQ ID NO: 16 - protein, 2 aa, artificial sequence
  Synthetic polypeptide
LQ